(19) United States
(12) Patent Application Publication
(10) Pub. No.: US 2014/0348756 A1
(43) Pub. Date: Nov. 27, 2014

(54) WATER-FREE FORMULATIONS HAVING A COOLING EFFECT

(71) Applicant: Henkel AG & Co. KGaA, Duesseldorf (DE)

(72) Inventors: Thomas Doering, Dormagen (DE); Gertraud Teckenbrock, Sprockhoevel (DE); Sandra Mausberg, Haan (DE); Daniel Solich, Langenfeld (DE)

(73) Assignee: Henkel AG & Co. KGaA, Duesseldorf (DE)

(21) Appl. No.: 14/222,213

(22) Filed: Mar. 21, 2014

Related U.S. Application Data
(63) Continuation of application No. PCT/EP2012/068517, filed on Sep. 20, 2012.

(30) Foreign Application Priority Data
Sep. 23, 2011 (DE) 10 2011 083 293.9

(51) Int. Cl. A61K 8/97; A61Q 19/00; A61Q 15/00; A61Q 11/00; A61K 8/34; A61K 8/26

(52) U.S. Cl. CPC A61K 8/97; A61K 8/347; A61K 8/26; A61Q 15/00; A61Q 11/00; A61Q 19/00
USPC 424/43; 424/774; 424/68

(57) ABSTRACT

Compositions for personal hygiene or body care, in particular deodorants, antiperspirants, massage fluids, foot care agents, shaving aids, or oral care agents, which preferably are present as a spray, including 0-7 wt % of free water, at least one cosmetic oil that is liquid under normal conditions, an oily extract from the leaves of Rumex acetosa, and in addition to the aforementioned components at least one cooling agent, wherein the amount specifications are relative to the weight of the composition without taking into account any present propellant.

### FIELD OF THE INVENTION

The present invention generally relates to compositions for personal hygiene and body care which engender a cooling sensorial impression.

### BACKGROUND OF THE INVENTION

Antiperspirants and deodorants in aerosol form or spray form, when applied, have a cooling and hence refreshing effect. However, the desired cooling effect only lasts for some seconds.

In order to achieve a longer-lasting, cooling sensory effect in compositions for personal hygiene and body care, use is typically made of menthol, in particular (−)-menthol, or selected menthol derivatives, for example menthyl lactate. Menthol, due to its relatively strong inherent smell and inherent taste, has limited use in perfuming or flavoring cosmetics because it can be combined only with a limited number of fragrances and aromas. In order to be perceived by the majority of consumers, higher concentrations of menthol and cooling menthol derivatives are required, particularly on the skin. In the quantities that are used for the desired intensive sensorial impression on the skin, particularly on the skin in the region of the armpit, menthol and cooling menthol derivatives can cause unpleasant side effects such as burning, itching and a feeling of dampness. With greater amounts, an unwanted feeling of dampness up to increased perspiration has been observed. Menthol can also impair skin compatibility of the product in the armpit area.

It is therefore desirable to provide cosmetic compositions, in particular deodorants, antiperspirants, massage fluids, foot care agents, shaving auxiliaries or oral care agents, with a long-lasting cooling sensory impression, and which possess good skin compatibility.

It is also desirable to provide cosmetic compositions, in particular deodorants, antiperspirants, massage fluids, foot care agents, shaving auxiliaries or oral care agents, with a long-lasting cooling sensory impression, wherein the cooling agent or the cooling agent combination has the lowest possible inherent smell or inherent taste.

It is further desirable to provide an active substance to produce a cooling sensory impression which can be incorporated into a cosmetic composition, in particular a deodorant, antiperspirant, massage fluid, foot care agent, shaving auxiliary or oral care agent, in order to prolong the cooling sensory effect of L-menthol or of another cooling agent.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A composition for personal hygiene or body care which is preferably a deodorant, antiperspirant, massage fluid, foot care agent, shaving auxiliary or oral care agent, comprising

a) 0-7 wt % free water,

b) at least one cosmetic oil that is liquid under normal conditions,

c) an oily extract from the leaves of Rumex acetosa,

d) in addition to the abovementioned components one or more cooling agents,

wherein all indications on quantities refer to the weight of the composition, without taking into account optionally present propellant.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In the course of investigating possible alternative cooling agents it was surprisingly found that an oily extract from the leaves of Rumex acetosa (=sorrel) produces a cooling sensory impression in the armpit, and which is comparable with that of (−)-menthol.

It was also surprisingly found that the combination of L-menthol with an oily extract from the leaves of Rumex acetosa makes the cooling effect of the L-menthol perceptible for longer, without causing negative side effects.

The designations (−)-menthol and L-menthol are used synonymously in the present application.

The subject matter of the present invention is a composition for personal hygiene or body care which is preferably a deodorant, antiperspirant, massage fluid, foot care agent, shaving auxiliary or oral care agent comprising 0-7 wt % free water, at least one cosmetic oil that is liquid under normal conditions, an oily extract from the leaves of Rumex acetosa, in addition to the abovementioned components one or more cooling agents, wherein all indications on quantities refer to the weight of the composition, without taking into account optionally present propellant.

A further subject matter of the present invention is the use of an oily extract from the leaves of Rumex acetosa for prolonging and/or boosting the cooling effect of a cooling agent, such as for example L-menthol. A further subject matter of the present invention is the use of an oily extract from the leaves of Rumex acetosa for prolonging and/or boosting the cooling effect of a cooling agent, such as for example L-menthol, in a composition for personal hygiene or body care which comprises 0-7 wt % free water, based on the weight of the composition, without taking into account optionally present propellant.

The inventively used oily extract from the leaves of Rumex acetosa can be obtained by extraction from leaves of Rumex acetosa with a cosmetic oil, preferably at a temperature of 5° C. to 30° C., particularly preferably 10° C. to 25° C. The term oily extract means that an oil was used as the extracting agent. The inventively used extract itself can be a powder, therefore does not have to be liquid itself under normal conditions or be dissolved or suspended in an oil. The extraction is particularly preferably effected with circulating oil, preferably at a temperature of 5° C. to 30° C., further exceptionally preferably 10° C. to 25° C. The cosmetic oil used for the extraction is preferably at least one oil that is liquid under normal conditions, which is selected from the esters of linear or branched C₁-C₂₂ alkanols or C₁₄-C₂₂ alkenols or from the triesters of glycerin with linear or branched, saturated or unsaturated C₂-C₂₂ carboxylic acids. Particularly preferred extraction oils are selected from isopropyl palmitate, isopropyl stearate, isopropyl myristate, caprylic/capric triglycerides, triesters of glycerin with n-octanoic acid and n-decanoic acid as well as with mixtures of n-octanoic acid and n-decanoic acid, 2-hexyldecyl stearate, hexyldecyl laurate, isononyl isononanoate, 2-ethylhexyl palmitate and 2-ethylhexyl stearate as well as mixtures thereof. Likewise preferred are isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-hexyl laurate, n-decyl oleate, oleyl oleate, oleyl erucate, erucyl oleate, C₁₂-C₁₅ alkyl lactate and di-C₁₂-C₁₃ alkyl malate as well as the benzoic acid esters of linear or branched C₈₋₂₂ alkanols as well as mixtures thereof. Isopropyl palmitate and isopropyl myristate as well as their mixtures are exceedingly preferred.

In regard to the inventively used cosmetic oils b) it should be noted that some esters of linear or branched C₁-C₂₂ alkanols or C₁₄-C₂₂ alkenols and some triesters of glycerin with linear or branched C₂-C₂₂ carboxylic acids that may be saturated or unsaturated, are solid under normal conditions, such as for example cetyl stearate or glycerin stearate (=stearin). These esters that are solid under normal conditions do not inventively represent cosmetic oils b), as they do not in fact fulfil the condition “liquid under normal conditions”. The attribution of whether an ester of this kind is liquid or solid under normal conditions remains within the scope of the general knowledge of the person skilled in art.

Preferred compositions according to the invention for personal hygiene or body care comprise the oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention, without taking into account optionally present propellant.

An inventively preferred oily extract from the leaves of Rumex acetosa is available from the Botanica Company under the name “Sorrel Extract in Isopropyl Myristate”.

A further subject matter of the present invention are compositions for personal hygiene or body care, in particular deodorants or antiperspirants, which are preferably in the form of a spray, comprising 0-7 wt % free water, at least one cosmetic oil that is liquid under normal conditions, an oily extract from the leaves of Rumex acetosa, in addition to the abovementioned components one or more cooling agents, in a total amount of 0.01-5 wt %, preferably in a total amount of 0.05-2 wt %, particularly preferably in a total amount of 0.1 to 1 wt %, exceedingly preferably in a total amount of 0.2-0.6 wt %, wherein the indications on quantities refer to the weight of the composition, without taking into account optionally present propellant.

Cooling agents are inventively regarded as those compounds that, similarly to L-menthol, stimulate the thermal receptors in the skin and in the mucous membranes, such that there results a cooling sensory impression. In particular, the receptor CMR-1 (cold and menthol-sensitive receptor) that belongs to the family of the TRP-channels (Transient Receptor Potential Ion Channels) is stimulated by the cooling agent, such that a feeling of cold is perceived. CMR-1 is expressed in certain peripheral neurons. The cooling agent binds onto the CMR-1 receptor that is also called TRPM-8 (Transient Receptor Potential Melastatin 8). This binding, similarly to low temperatures, causes the ion channel to open, such that calcium and sodium ions pass through, thereby resulting in a feeling of cold at the end of the signal chain.

Inventively preferred cooling agents are selected from menthol, preferably (−)-menthol, additionally from isopulegol, in particular (−)-isopulegol (FEMA 2962), N-2,3-trimethyl-2-isopropylbutamide, menthyl lactate, menthylpyrrolidone carboxylic acid, menthyl methyl ether, menthoxypropane-1,2-diol, menthone glycerin acetal (9-methyl-6-(1-methylethyl)-1,4-dioxaspiro(4.5)decane-2-methanol), monomenthyl succinate, menthyl ethylamido oxalate (CAS-nr. 1122460-01-8), 2-hydroxy-methyl-3,5,5-trimethylcyclohexanol, 2-isopropyl-N,2,3-trimethylbutyramide (FEMA 3804), N-ethyl-p-menthane-3-carboxamide (FEMA 3455), in particular 1R,3R,4S—N-ethyl-p-menthane-3-carboxamide, ethyl 3-(p-menthane-3-carboxamido) acetate (FEMA 4309), (1R,2S,5R)-n-(4-methoxyphenyl)-p-menthane carboxamide (FEMA 4681), N-ethyl-2,2-diisopropylbutanamide (FEMA 4557), N-cyclopropyl-5-methyl-2-propane-2-yl-cyclohexane-1-carboxamide (FEMA 4693), N-(4-cyanomethylphenyl)-p-menthane carboxamide (FEMA 4496), N-(2-(pyridin-2-yl)ethyl)-3-p-menthane carboxamide (FEMA 4549), N-(2-hydroxyethyl)-2-isopropyl-2,3-dimethylbutanamide (FEMA 4602), N-(1,1-dimethyl-2-hydroxyethyl)-2,2-diethylbutanamide (FEMA 4603), (2S,5R)—N-[4-(2-amino-2-oxoethyl)phenyl]-p-menthane carboxamide (FEMA 4684), 2-[(2-p-menthoxy)-ethoxy]ethanol (FEMA 4718), (2,6-diethyl-5-isopropyl-2-methyltetrahydropyran (FEMA 4680), 3-(1-menthoxy)-2-methylpropane-1,2-diol (FEMA 3849), p-menthane-3,8-diol, in particular (+)-cis-p-menthane-3,8-diol and (−)-trans-p-menthane-3,8-diol as well as mixtures of (+)-cis-p-menthane-3,8-diol and (−)-trans-p-menthane-3,8-diol, in particular a mixture in the weight ratio 62:38 (FEMA 4053), (1R,3R,4S)-3-menthyl 3,6-dioxaheptanoate, (1R,2S,5R)-3-menthyl-methoxy acetate, (1R,2S,5R)-3-menthyl 3,6,9-trioxadecanoate, (1R,2S,5R)-3-menthyl 3,6,9-tri-oxadecanoate, (1R,2S,5R)-3-menthyl-(2-hydroxyethoxy) acetate, (1R,2S,5R)-menthyl 11-hydroxy-3,6,9-trioxaundecanoate, (−)-cubebol (FEMA 4497), N-(4-cyanomethylphenyl)-p-menthane carboxamide, N,N-dimethylmenthylsuccinamide(2-isopropyl-5-methylcyclohexyl 4-(dimethylamino)-4-oxobutanoate, FEMA 4230), 6-isopropyl-3,9-dimethyl-1,4-dioxaspiro[4.5]decane-2-one (FEMA 4285), as well as mixtures of these substances, wherein (−)-menthol is particularly preferred. L-Menthol is extremely preferred. In so far as the abovementioned compounds are not described in more detail in regard to their stereoisomers, the all-equatorial isomers of the abovementioned compounds may be regarded as preferred.

For the sake of clarity it should be noted that the oily extract from the leaves of Rumex acetosa does not fall under the term cooling agent and is not taken into account when calculating the amount of cooling agent.

Preferred compositions according to the invention comprise one or more cooling agents in a total amount of 0.01-5 wt %, preferably in a total amount of 0.05-2 wt %, particularly preferably in a total amount of 0.1 to 1 wt %, exceedingly preferably in a total amount of 0.2-0.6 wt %, wherein the quantities refer to the weight of the composition, without taking into account optionally present propellant.

Other preferred compositions according to the invention are characterized in that the weight ratio of cooling agent(s) to dry extract of the oily extract from the leaves of Rumex acetosa is 0.01-200, preferably 0.1-100, particularly preferably 1-60, exceptionally preferably 10-50.

Particularly preferred inventive compositions for personal hygiene or body care, in particular deodorants or antiperspirants, comprise 0-7 wt % free water, at least one cosmetic oil that is liquid under normal conditions, an oily extract from the leaves of Rumex acetosa, in addition to the abovementioned components at least one cooling agent; the compositions being packaged in a spray can with a propellant and optional other active substances and/or auxiliaries, wherein the indications on quantities refer to the weight of the composition, without taking into account optionally present propellant.

The inventive compositions are essentially anhydrous, i.e. they comprise 0 to maximum 7 wt %, maximum 0.5-5 wt %, particularly preferably 1 to 4 wt %, exceedingly preferably 1.5 to 3 wt % of free water, wherein contents of free water can also preferably be 1.6-2.4 wt %, 1.7-2.3 wt % and 1.8-2.2 wt % and wherein the indications on quantities refer to the weight of the composition, without taking into account optionally present propellant. The content of water of crystallization, water of hydration or similar molecularly bound water which is comprised in the added ingredients, in particular in the antiperspirant active substances, does not represent free water in the context of the present application.

In the context of the present application, normal conditions are a temperature of 20° C. and a pressure of 1013.25 mbar. Melting point data likewise refer to a pressure of 1013.25 mbar.

The inventive compositions comprise at least one cosmetic oil.

Preferred inventive compositions are antiperspirant compositions, in particular based on a suspension that is sprayable with or without propellant. Other preferred inventive compositions are deodorant compositions, in particular based on a solution that is sprayable with or without propellant. Inventive compositions made up as suspensions and solutions can be applied not only as a spray but also for example as a roll-on. Through its cooling effect, the inventively used combination of the oily extract from Rumex acetosa leaves and cooling agent bolsters the perception of the antiperspirant or deodorizing effect of such products.

The inventive compositions that are not applied as aerosols are preferably made up according to requirements of the type of application.

Preferred inventive non-aerosol compositions are in the form of solids, semi-solids, liquids, dispersions, anhydrous emulsions, suspensions or gels.

In a particularly preferred embodiment, the inventive compositions exist in liquid form. In the context of the invention, the term liquid also includes any solid dispersions in liquids. Other inventively preferred compositions can also be pastes, ointments, lotions or creams. Inventively preferred solid compositions can be for example a loose powder, compressed powder or a stick. Inventively preferred compositions in stick form, in particular deodorant or antiperspirant sticks, can be in the form of a gel, wherein the oil phase comprises a lipophilic thickener or solidifier, such as waxes and/or alcohols that are solid under normal conditions, in particular cetyl alcohol and/or stearyl alcohol. Compositions in the form of creams, gels, pastes and liquids can be packaged e.g. in pump dispensers or squeeze dispensers, in particular in multi-chamber pump dispensers or multi-chamber squeeze dispensers. In the case of e.g. liquid agents, preferably they can also be applied with a roller applicator, as is known for example from the field of deo-rollers. Rollers of this type have a ball located in a ball bed, whereby the ball can be moved over a surface. The ball takes up some of the agent to be spread on and conveys it onto the surface to be treated.

The application can also be made e.g. with substrates that are supplied with an inventive preparation. Wet wipes are particularly preferred, i.e. prepackaged, preferably individually packaged, wet wipes for the user, as are well known from the field of glass cleaning (eye glass cleaning cloths) or from the field of moist toilet paper. Wet wipes of this kind which can also preferably comprise preservatives, are then impregnated or supplied with an inventive agent. They can be employed for example as a deodorant cloth, this being particularly interesting for use when travelling. It can be particularly preferred when these cloths are individually packaged. Inventive agents, preferably liquid agents, can also be in a multi-phase form; the phases can be arranged e.g. horizontally, i.e. one on top of the other, or vertically, i.e. beside one another. It can also be a dispersed system, in which e.g. the solid ingredients are inhomogeneously distributed in the liquid matrix, such that a disperse system of this kind should be shaken before use.

Furthermore, the inventive compositions that are formulated as deodorant or antiperspirant sticks can be based on fats, based on a polyol in oil emulsion, based on an oil in polyol emulsion, based on a polyol-oil multiple emulsion, based on a nano-emulsion and based on a micro-emulsion, wherein the polyol phase can be anhydrous or have only a minor amount of free water (e.g. 0.5-7 wt %, preferably 1-6 wt %, each based on the total composition. Gel sticks can be formulated on the basis of alditols, in particular dibenzylidene sorbitol, N-acylamino acid amides, 12-hydroxystearic acid, polyamides and polyamide derivatives.

Inventively preferred compositions that are in the form of deodorant or antiperspirant wax sticks comprise 30-70 wt % of at least one cosmetic oil that is liquid under normal conditions and 15-25 wt % of a fatty component that is solid under normal conditions, from which the greater part usually has a melting point of ca. 50° C. or usually consists of fatty alcohols, in particular of stearyl alcohol, but also cetyl alcohol and optionally even arachidyl alcohol and/or behenyl alcohol, whereas a minor fraction—about 0.5-5 wt %—consists of at least one fatty component with a melting point of ca. 55-120° C. Moreover, 0.5-8 wt % of at least one fatty component having a melting point of ca. 25-35° C. may be comprised. In addition, 0.5-30 wt % of at least one filler may be comprised, typically selected from talc, cellulose powders, starch and starch derivatives. In addition, 0.1-10 wt %, preferably 1-5 wt %, particularly preferably 2-4 wt % of at least one oil in water emulsifier may be comprised.

Inventively preferred antiperspirant suspensions that can be sprayed with a propellant comprise at least one cosmetic oil as the carrier for the particulate antiperspirant active substance, in addition to antiperspirant active substances. These suspensions are filled in a pressure-resistant container, mostly a tin-plate or aluminum can that is lacquered inside, together with a liquefied hydrocarbon, such as n-butane, iso-butane and/or propane, as the propellant. Before using the spray valve that releases the propellant and a portion of the suspension, the container has first to be sufficiently shaken so as to mix up the settled antiperspirant. In order to prevent the suspended antiperspirant from immediately settling out again, customary suspensions comprise a suspension agent, e.g. hydrophobically modified Hectorite and Bentonite, as are available for example under the INCI names Disteardimonium Hectorite, Stearalkonium Hectorite, Stearalkonium Bentonite, Quaternium-18 Hectorite, Quaternium-18 Bentonite or Dihydrogenated Tallow Benzylmonium Hectorite.

The inventively used cooling agents are in fact lipophilic, and some of them are liquid under normal conditions, but are not inventively counted as the cosmetic oils b). Fragrances that are usually comprised in cosmetic compositions, are also in fact lipophilic, but are not inventively counted as the cosmetic oils b).

Inventively preferred compositions comprise cosmetic oils that are liquid under normal conditions in a total amount of 1-95 wt %, preferably 5-90 wt %, particularly preferably 30 to 75 wt %, exceptionally preferably 50-60 wt %, wherein the quantities refer to the weight of the composition, without taking into account optionally present propellant.

Inventively preferred compositions that are packaged as a sprayable suspension or solution or which can be applied with a roller ball, i.e. particularly for inventively preferred antiperspirant compositions, comprise cosmetic oils that are liquid under normal conditions in a total amount of 1-95 wt %, preferably 5-90 wt %, particularly preferably 10 to 85 wt %, exceptionally preferably 20-80 wt %, more preferably 30-75 wt %, also very preferably 50-70 wt %, also exceptionally preferably 54-64 wt %, wherein the quantities refer to the weight of the composition, without taking into account optionally present propellant. A total quantity of at least one cosmetic oil that is liquid under normal conditions of 60-80 wt % and 65-76 wt %, each relative to the total propellant-free composition, can also be inventively particularly preferred, wherein a total quantity of 69-74 wt %, relative to the total propellant-free composition, is particularly preferred.

Inventively preferred compositions that are packaged as sticks comprise a total amount of cosmetic oils that are liquid under normal conditions in 30-70 wt %, preferably 40-60 wt %, particularly preferably 45 to 55 wt %, exceptionally preferably 50-53 wt % of at least one cosmetic oil that is liquid under normal conditions, each relative to the total composition.

For the cosmetic oils, one differentiates between volatile and non-volatile oils. Non-volatile oils are understood to mean those oils that have a vapor pressure of less than 2.66 Pa (0.02 mm Hg) at 20° C. and an ambient pressure of 1013 hPa. Volatile oils are understood to mean those oils that have a vapor pressure of 2.66 Pa-40 000 Pa (0.02 mm-300 mm Hg), preferably 13-12 000 Pa (0.1-90 mm Hg), particularly preferably 15-8000 Pa, extremely preferably 300-3000 Pa, at 20° C. and an ambient pressure of 1013 hPa.

Volatile cosmetic oils are usually selected from silicone oils with the INCI name Cyclomethicones. Cyclomethicones under the INCI name are in particular understood to be cyclotrisiloxane (hexamethylcyclotrisiloxane), cyclotetrasiloxane (octamethylcyclotetrasiloxane), cyclopentasiloxane (decamethylcyclopentasiloxane) and cyclohexasiloxane (dodecamethylcyclohexasiloxane). These oils have a vapor pressure of ca. 13-15 Pa at 20° C.

In the prior art, cyclomethicones are known as very suitable oils for cosmetic compositions, in particular for antiperspirant and deodorant compositions, such as sprays and sticks. Due to their persistence in the environment it can, however, be preferred to refrain from using cyclomethicones. In a specifically preferred embodiment, the compositions according to the invention comprise 0 to less than 1 wt % of cyclomethicone, based on the weight of the composition, without taking into account optionally present propellant.

Other preferred volatile silicone oils are selected from volatile linear silicone oils, in particular volatile silicone oils including 2-10 siloxane units, such as hexamethyldisiloxane (L₂), octamethyltrisiloxane (L₃), decamethyltetrasiloxane (L₄), as are available for example in the commercial products DC 2-1184, Dow Corning® 200 (0.65 cSt) and Dow Corning® 200 (1.5 cSt) from Dow Corning, and low molecular weight Phenyl Trimethicone with a vapor pressure of about 2000 Pa at 20° C., as is available for example from GE Bayer Silicones/Momentive under the name Baysilone Fluid PD 5.

Preferred compositions according to the invention, particularly antiperspirant compositions, comprise at least one volatile silicone oil that may be cyclic or linear, due to the drier skin feeling and the faster release of active substance.

Other preferred compositions according to the invention, particularly antiperspirant compositions, comprise at least one volatile non-silicone oil, due to the drier skin feeling and the faster release of active substance Preferred volatile non-silicone oils are selected from C₈-C₁₆ isoparaffins, in particular from isononane, isodecane, isoundecane, isododecane, isotridecane, isotetradecane, isopentadecane and isohexadecane, as well as mixtures thereof. C₁₀-C₁₃ Isoparaffin mixtures are preferred, in particular those with a vapor pressure of about 300-400 Pa, preferably 360 Pa at 20° C. This at least one C₈-C₁₆ isoparaffin is preferably comprised in a total amount of 25-50 wt %, preferably 30-45 wt %, particularly preferably 32 to 40 wt %, exceptionally preferably 34-37 wt %, each relative to the total weight of the propellant-free composition.

In inventively preferred compositions the at least one oil b) that is liquid under normal conditions includes at least one volatile C₈-C₁₆ isoparaffin, in particular isononane, isodecane, isoundecane, isododecane, isotridecane, isotetradecane, isopentadecane and isohexadecane as well as mixtures thereof.

Further inventively preferred compositions comprise triethyl citrate and at least one C₈-C₁₆ isoparaffin, selected from isononane, isodecane, isoundecane, isododecane, isotridecane, isotetradecane, isopentadecane and isohexadecane as well as mixtures of these isoparaffins. Further inventively preferred compositions comprise triethyl citrate and at least one C₈-C₁₆ isoparaffin, selected from isononane, isodecane, isoundecane, isododecane, isotridecane as well as mixtures of these C₈-C₁₆ isoparaffins.

Further inventively preferred compositions comprise triethyl citrate and a mixture of isodecane, isoundecane, isododecane and isotridecane.

Further inventively preferred compositions, in particular antiperspirant compositions, comprise at least one non-volatile cosmetic oil, selected from non-volatile silicone oils and non-volatile non-silicone oils. Residues from insoluble ingredients in the carrier such as antiperspirants or talc can be successfully masked with a non-volatile oil. Moreover, parameters such as skin feel, visibility of the residue and stability of the inventive composition can be finely adjusted with a mixture of various oils, in particular a non-volatile and a volatile oil, and can be better adapted to the needs of the consumers.

It is also possible, of course, to formulate inventive compositions with a low level of volatile oils—that means with 0.5-24.5 wt %, relative to the weight of the propellant-free composition—or even without volatile oils.

Optionally hydroxylated esters of the linear or branched, saturated or unsaturated fatty alcohols including 2-30 carbon atoms with linear or branched, saturated or unsaturated fatty acids including 2-30 carbon atoms are inventively particularly preferred. Optionally hydroxylated esters of the linear or branched, saturated fatty alcohols including 2-5 carbon atoms with linear or branched, saturated or unsaturated fatty acids including 10-18 carbon atoms are preferred. Isopropyl palmitate, isopropyl stearate, isopropyl myristate, 2-hexyldecyl stearate, 2-hexyldecyl laurate, isononyl isononanoate, 2-ethylhexyl palmitate and 2-ethylhexyl stearate are preferred examples. Isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-hexyl laurate, n-decyl oleate, oleyl oleate, oleyl erucate, erucyl oleate, C₁₂-C₁₅ alkyl lactate and di-C₁₂-C₁₃ alkyl malate as well as the benzoic acid esters of linear or branched C₈₋₂₂ alkanols are likewise preferred. C₁₂-C₁₅ Alkyl esters of benzoic acid, e.g. available as the commercial product Finsolv® TN (C₁₂-C₁₅ alkyl benzoate), as well as the isostearyl ester of benzoic acid, e.g. available as Finsolv® SB, 2-ethylhexyl benzoate, e.g. available as Finsolv® EB, and the 2-octyldodecyl ester of benzoic acid, e.g. available as Finsolv® BOD are particularly preferred. Another particularly preferred ester oil is triethyl citrate.

Inventively preferred oil mixtures are triethyl citrate/2-ethylhexyl palmitate, triethyl citrate/hexyldecyl laurate, triethyl citrate/2-ethylhexyl stearate, triethyl citrate/isopropyl myristate, triethyl citrate/isopropyl palmitate, triethyl citrate/2-ethylhexyl laurate, triethyl citrate/C₁₂-C₁₅ alkyl lactate, triethyl citrate/C₁₂-C₁₅ alkyl benzoate and triethyl citrate/di-C₁₂-C₁₃ alkyl malate. Particularly preferred oil mixtures are triethyl citrate/isopropyl myristate, triethyl citrate/isopropyl palmitate, triethyl citrate/2-ethylhexyl palmitate and triethyl citrate/C₁₂-C₁₅ alkyl benzoate.

Other inventively preferred non-volatile non-silicone oils are selected from branched, saturated or unsaturated fatty alcohols including 6 to 30 carbon atoms. These alcohols are often called Guerbet alcohols as they are obtained by the Guerbet reaction. Preferred alcohol oils are 2-hexyldecanol, 2-octyldodecanol and 2-ethylhexyl alcohol. Isostearyl alcohol is likewise preferred. Other preferred non-volatile oils are selected from mixtures of Guerbet alcohols and esters of Guerbet alcohols, e.g. 2-hexyldecanol and 2-hexyldecyl laurate.

The term triglyceride used in the following means triesters of glycerin. Other inventively preferred non-volatile oils are selected from the triglycerides of linear or branched, saturated or unsaturated, optionally hydroxylated C₈₋₃₀ fatty acids, in so far as they are liquid under normal conditions. The use of naturally occurring oils can be particularly suitable, for example soya oil, cotton seed oil, sunflower oil, palm oil, palm kernel oil, linseed oil, almond oil, castor oil, corn oil, rapeseed oil, olive oil, sesame oil, safflower oil, wheat germ oil, peach kernel oil and the liquid fractions of cocoa oil and the like. Synthetic triglyceride oils are particularly preferred, in particular capric/caprylic triglycerides, e.g. the commercial products Myritole® 318 or Myritolo® 331 (BASF/Cognis) with unbranched fatty acid residues as well as glyceryl triisostearin and glyceryl tri(2-ethylhexanoate) with branched fatty acid residues. These types of triglyceride oils preferably make up a fraction of less than 50 wt % of the total weight of all cosmetic oils in the inventive composition. The total weight of triglyceride oils is particularly preferably 0.5-10 wt %, preferably 1-5 wt %, each relative to the total composition, without taking into account optionally present propellant.

Other inventively particularly preferred non-volatile non-silicone oils are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Other inventively particularly preferred non-volatile non-silicone oils are selected from the addition products of 1 to 5 propylene oxide units onto monohydric or polyhydric C₈₋₂₂ alkanols such as octanol, decanol, decane diol, lauryl alcohol, myristyl alcohol and stearyl alcohol, e.g. PPG-2-myristyl ether and PPG-3-myristyl ether.

Other inventively particularly preferred non-volatile non-silicone oils are selected from the addition products of at least 6 ethylene oxide units and/or propylene oxide units onto monohydric or polyhydric C₃₋₂₂ alkanols such as glycerin, butanol, butane diol, myristyl alcohol and stearyl alcohol, which can be optionally esterified, e.g. PPG-14-butyl ether, PPG-9-butyl ether, PPG-10-butane diol, PPG-15-stearyl ether and glycereth-7-diisononanoate.

Other inventively particularly preferred non-volatile non-silicone oils are selected from the symmetric, unsymmetric or cyclic esters of carbonic acid with C₆-C₂₀ alcohols, e.g. di-n-caprylyl carbonate (Cetiol® CC) or di-(2-ethylhexyl) carbonate (Tegosoft DEC). In contrast, esters of carbonic acid with C₁-C₅ alcohols, e.g. glycerin carbonate or propylene carbonate, are not suitable compounds as the cosmetic oil d). Nonetheless, propylene carbonate can be comprised in the inventive compositions, primarily as an activator for the lipophilic thickener, in particular for hydrophobically modified Bentonite and Hectorite. Propylene carbonate and other esters of carbonic acid with C₁-C₅ alcohols are not taken into account for the calculation of the total quantity of oils b).

Other oils that can be inventively preferred are selected from the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols. The total weight of dimer fatty acid esters is particularly preferably 0.5-10 wt %, preferably 1-5 wt %, each relative to the total composition, without taking into account optionally present propellant.

Another inventively preferred oil b) is triethyl citrate. Particularly preferred oils b) consist of mixtures of the abovementioned oils. It is also particularly preferred to use mixtures of triethyl citrate and at least one optionally hydroxylated ester of the linear or branched, saturated or unsaturated fatty alcohols including 2-30 carbon atoms with linear or branched, saturated or unsaturated fatty acids including 2-30 carbon atoms. In such mixtures, the ethyl esters and the isopropyl esters are particularly preferred; isopropyl palmitate and isopropyl myristate are exceedingly preferred. Another inventively particularly preferred oil mixture is triethyl citrate/2-ethylhexyl palmitate.

The antiperspirant active substances and optional additional active substances that are insoluble in the oil b) are suspended in the at least one oil b) that is liquid under normal conditions. If a suspension of this type is intended to be sprayed on or applied as a roll on, then at least one lipophilic thickener is preferably added to this suspension as a suspension aid for better applicability. When producing a stick, usually a wax ester and/or a linear alcohol that includes at least 12 carbon atoms, each being solid under normal conditions, are added as the lipophilic thickener. Other preferred inventive compositions therefore comprise at least one lipophilic thickener.

Preferred inventive compositions comprise at least one lipophilic thickener that is selected from hydrophobized mineral clays, pyrogenic silicas, bentone gels, ethylene/propylene/styrene copolymers, butylene/ethylene/styrene copolymers, dextrin esters, silicone elastomers, waxes that are solid under normal conditions and/or glycerin triesters that are solid under normal conditions and/or alkanols that are solid under normal conditions. Among these, hydrophobized mineral clays are particularly preferred. Preferred hydrophobized mineral clays are selected from hydrophobized Montmorillonites, hydrophobized Hectorites and hydrophobized Bentonites, particularly preferably from Disteardimonium Hectorite, Stearalkonium Hectorite, Stearalkonium Bentonite, Quaternium-18 Hectorite, Quaternium-18 Bentonite and Dihydrogenated Tallow Benzylmonium Hectorite. The commercial thickeners provide these hydrophobized mineral clays as powders or in the form of a gel in an oil component. These types of powders or gels are available for example under the trade names Bentone® or Thixogel.

Inventively preferred compositions are characterized in that they comprise at least one hydrophobized mineral clay in a total amount of 0.5-10 wt %, preferably 1-7 wt %, particularly preferably 2-6 wt %, exceptionally preferably 3-5 wt %, each relative to the total weight of the propellant-free composition.

These types of hydrophobized mineral clays usually require water, ethanol or propylene carbonate as an activator in an amount of 0.3-3 wt %, preferably 0.5 to 2 wt %, each relative to the total weight of the propellant-free inventive composition.

Other inventively preferred lipophilic thickeners are selected from pyrogenic silicas, e.g. the commercial products of the Aerosil® series from Evonik Degussa. Hydrophobized pyrogenic silicas are particularly preferred and silica silylates and silica dimethyl silylates are exceedingly preferred.

Inventively preferred compositions are characterized in that they comprise at least one pyrogenic silica, preferably at least one hydrophobized pyrogenic silica, in a total amount of 0.5-10 wt %, preferably 0.8-5 wt %, particularly preferably 1-4 wt %, exceptionally preferably 1.5-2 wt %, each relative to the total weight of the propellant-free inventive composition.

Other inventively preferred compositions are characterized in that they comprise at least one hydrophobized pyrogenic silica and at least one hydrophilic silica.

Other inventively preferred compositions, in particular those in the form of sticks and so-called soft solids (=sticks with a creamy-like solidity), are characterized in that they comprise at least one fatty component that is solid under normal conditions with a melting point greater than 50-120° C.

Preferred fatty components with a melting point of >50-120° C. are selected from waxes. Waxes are generally of a solid to a hard and brittle consistency, coarse to fine crystalline, translucent to opaque, but not glassy, and melt above 50° C. without decomposition. Already slightly above the melting point they have a low viscosity and show a strongly temperature-dependent consistency and solubility.

Naturally-occurring vegetal waxes for example are inventively preferred, e.g. candelilla wax, carnauba wax, Japan wax, sugar cane wax, ouricoury wax, cork wax, sunflower wax, fruit waxes such as orange waxes, lemon waxes, grapefruit wax, and animal waxes, e.g. beeswax, shellac wax and spermaceti. In the context of the invention, it can be particularly preferred to employ hydrogenated or hardened waxes. Chemically modified waxes, in particular the hard waxes, such as e.g. Montan ester waxes, hydrogenated jojoba waxes and Sasol waxes can also be employed as the wax component. The synthetic waxes that are likewise inventively preferred include for example polyalkylene waxes, in particular polyethylene waxes, polyalpha-olefin and polyethylene glycol waxes, C₂₀-C₄₀ dialkyl esters of dimeric acids, C₃₀₋₅₀ alkyl beeswax as well as alkyl and alkylaryl esters of dimer fatty acids.

A particularly preferred wax component is selected from at least one ester of a saturated, monohydric C₁₆-C₆₀ alcohol and a saturated C₈-C₃₆ monocarboxylic acid. Lactides, the cyclic double esters of α-hydroxycarboxylic acids of the corresponding chain length, are also inventively included here Esters of fatty acids and long chain alcohols have proven to be particularly advantageous for the inventively preferred compositions, because they lend excellent sensory properties and a high stability to the sticks. The esters are constituted from saturated, branched or unbranched monocarboxylic acids and saturated, branched or unbranched monohydric alcohols. Esters of aromatic carboxylic acids or hydroxycarboxylic acids (e.g. 12-hydroxystearic acid) and saturated, branched or unbranched alcohols can also be employed, in so far as the wax component has a melting point>50° C. It is particularly preferred to select the wax components from the group of the esters of saturated, branched or unbranched alkane carboxylic acids with a chain length of 12 to 24 carbon atoms and the saturated, branched or unbranched alcohols with a chain length of 16 to 50 carbon atoms, wherein the wax components have a melting point>50° C.

In particular, C₁₆₋₃₆ alkyl stearates and C₁₈₋₃₈ alkylhydroxystearoyl stearates, C₂₀₋₄₀ alkyl erucates as well as cetearyl behenate can be preferred as the wax component. The wax or the wax components have a melting point>50° C. to 120° C., preferably >60° C. to 100° C. A particularly preferred embodiment of the invention comprises a C₂₀-C₄₀ alkyl stearate as the wax component. This ester is known by the name Kesterwachs® K82H or Kesterwachs® K80H and is marketed by Koster Keunen Inc. Another particularly preferred embodiment of the invention comprises cetearyl behenate as the wax component, i.e. mixtures of cetyl behenate and stearyl behenate. This ester is known by the name Kesterwachs® K62 and is marketed by Koster Keunen Inc.

Other preferred wax components with a melting point>50° C. are the triglycerides of saturated and optionally hydroxylated C₁₂₋₃₀ fatty acids, such as hardened triglyceride fats (hydrogenated palm oil, hydrogenated cocoa oil, hydrogenated castor oil), glyceryl tribehenate (tribehenin) or glyceryl tri-12-hydroxystearate, additionally synthetic full esters of fatty acids and glycols or polyols including 2-6 carbon atoms, as long as they have a melting point above 50° C., for example preferably C₁₈₋₃₆ acid triglycerides (e.g. Syncrowax® HGL-C).

Hydrogenated castor oil, available for example as the commercial product Cutina® HR, is inventively particularly preferred as the wax component.

Other preferred wax components with a melting point>50° C. are the saturated linear C₁₄-C₃₆ carboxylic acids, in particular myristic acid, palmitic acid, stearic acid and behenic acid as well as mixtures of these compounds, e.g. Syncrowaxe® AW1C (C₁₈-C₃₆ fatty acids) or Cutina® FS 45 (palmitic and stearic acids).

Other preferred wax components with a melting point>50° C. to 120° C. are polyalpha-olefins and/or polyethylene.

Preferred inventive compositions comprise at least one wax component that is selected from esters of a saturated, monohydric C₁₆-C₆₀ alkanol and a saturated C₈-C₃₆ monocarboxylic acid, in particular cetyl behenate, stearyl behenate and C₂₀-C₄₀ alkyl stearate, glycerin triesters of saturated linear C₁₂-C₃₀ carboxylic acids that can be hydroxylated, candelilla wax, carnauba wax, beeswax, saturated linear C₁₄-C₃₆ carboxylic acids as well as mixtures of the cited substances. Particularly preferred mixtures of wax components are selected from mixtures of cetyl behenate, stearyl behenate, hydrogenated castor oil, palmitic acid and stearic acid. Other particularly preferred mixtures of wax components are selected from mixtures of C₂₀-C₄₀ alkyl stearate, hydrogenated castor oil, palmitic acid and stearic acid.

Particularly preferred inventive compositions comprise at least one wax component that is selected from mixtures of esters of a saturated, monohydric C₁₆-C₆₀ alkanol and a saturated C₈-C₃₆ monocarboxylic acid, in particular C₂₀-C₄₀ alkyl stearate, glycerin triesters of saturated linear C₁₂-C₃₀ carboxylic acids that can be hydroxylated, in particular hydrogenated castor oil, and saturated linear C₁₄-C₃₆ carboxylic acids, in particular palmitic acid and stearic acid.

Other preferred inventive compositions comprise at least one wax component in a total amount of 1-10 wt %, preferably 1.5-8 wt %, particularly preferably 2 to 6 wt %, exceptionally preferably 3-5 wt %, each relative to the total composition.

Other preferred inventive compositions comprise at least one wax component with a melting point in the range 25-50° C., selected from coco fatty acid glycerin mono, di and triesters, Butyrospermum Parkii (Shea Butter) and esters of saturated, monohydric C₈-C₁₈ alcohols with saturated C₁₂-C₁₈ monocarboxylic acids as well as mixtures of these substances. These low melting wax components enable the consistency of the product to be optimized and the visible residues on the skin to be minimized. Commercial products with the INCI name Cocoglycerides are particularly preferred, particularly preferably a mixture of C₁₂-C₁₈ mono, di and triglycerides, which melts in the range 30-32° C., as can be obtained for example under the trade name Novata® AB from Cognis, as well as the products of the Softisan series (Sasol Germany GmbH) with the INCI name Hydrogenated Cocoglycerides, in particular Softisan 100, 133, 134, 138, 142. Other preferred esters of saturated, monohydric C₁₂-C₁₈ alcohols with saturated C₁₂-C₁₈ monocarboxylic acids are stearyl laurate, cetearyl stearate (e.g. Crodamol® CSS), stearyl stearate (e.g. Estol 3706), cetyl palmitate (e.g. Cutina® CP, melting point: 46-50° C.) and myristyl myristate (e.g. Cetiol® MM, melting point: 38-42° C.).

Other preferred inventive compositions are characterized in that at least one wax component with a melting point in the range 25-50° C. is comprised in a total amount of 0.01-10 wt %, preferably 0.5-8 wt %, particularly preferably 1 to 7.5 wt %, exceptionally preferably 1.8-7 wt %, further preferably 2-5 wt %, as well as 3-4 wt %, each relative to the total composition.

Other inventively preferred compositions comprise at least one oil in water emulsifier. Preferred oil in water emulsifiers have an HLB value of more than 7. Other preferred oil in water emulsifiers are non-ionic. They concern emulsifiers that are generally known to the person skilled in the art and are listed for example in Kirk-Othmer, “Encyclopedia of Chemical Technology”, 3rd ed., 1979, Vol 8, pp. 913-916. The HLB value for ethoxylated products is calculated with the Formula HLB=(100−L): 5, wherein L is the weight fraction of the lipophilic groups, that is the fatty alkyl or fatty acyl groups, in the ethylene oxide adducts, expressed in weight percent.

When inventively choosing suitable non-ionic oil in water emulsifiers it is particularly preferred to incorporate a mixture of non-ionic oil in water emulsions, in order to be able to optimally adjust the properties of the inventive compositions, such as active substance release (in particular the release of an antiperspirant active substance) or washability. The individual emulsifier components here supply a proportion of the total HLB value or the average HLB value of the oil in water emulsifier mixture according to their quantity by weight in the total amount of the oil in water emulsifiers. However, in another preferred embodiment, the inventive compositions, in particular the deodorant or antiperspirant sticks can also comprise only one single oil in water emulsifier, preferably with an HLB value in the range 11-17, particularly preferably 12-15 and exceedingly preferably 13-14.

Preferred inventive cosmetic compositions comprise at least one non-ionic oil in water emulsifier that is selected from ethoxylated C₈-C₂₄ alkanols with an average of 10-100 moles ethylene oxide per mole, ethoxylated C₉-C₂₄ carboxylic acids with an average of 10-100 moles ethylene oxide per mole, ethoxylated sorbitol monoesters of linear saturated and unsaturated C₁₂-C₃₀ optionally hydroxylated carboxylic acids with on average 20-100 moles ethylene oxide per mole, in particular the acids being myristic acid, palmitic acid, stearic acid or mixtures of these fatty acids, silicone copolyols with ethylene oxide units or with ethylene oxide and propylene oxide units, alkyl mono and oligoglycosides with 8 to 22 carbon atoms in the alkyl group and their ethoxylated analogs, ethoxylated sterols, partial esters of polyglycerins with n=2 to 10 glycerin units and esterified with 1 to 4 saturated or unsaturated, linear or branched, optionally hydroxylated C₈-C₃₀ fatty acids, in so far as they have an HLB value of more than 7, as well as mixtures of the abovementioned substances.

The ethoxylated C₉-C₂₄ alkanols have the Formula R¹O(CH₂CH₂O)ₙH, wherein R¹ stands for a linear or branched alkyl and/or alkenyl group including 8-24 carbon atoms and n, the average number of the ethylene oxide units per molecule, for numbers from 10-100, preferably 10-30 mol ethylene oxide on 1 mol capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol as well as their technical mixtures. Adducts of 10-100 mol ethylene oxide on technical fatty alcohols with 12 to 18 carbon atoms are also suitable, such as for example coco, palm, palm kernel or tallow fatty alcohol.

The ethoxylated C₈-C₂₄ carboxylic acids have the Formula R¹O(CH₂CH₂O)ₙH, wherein R¹O stands for a linear or branched, saturated or unsaturated acyl group including 8-24 carbon atoms and n, the average number of the ethylene oxide units per molecule, for numbers from 10-100, preferably 10-30 mol ethylene oxide on 1 mol caprylic acid, 2-ethylhexanoic acid, capric acid, lauric acid, isotridecanoic acid, myristic acid, palmitic acid, palmitoleic acid, stearic acid, isostearic acid, öleic acid, elaidic acid, petroselic acid, arachydronic acid, gadoleic acid, behenic acid, erucic acid and brassidic acid as well as their technical mixtures. Adducts of 10-100 mol ethylene oxide on technical fatty acids with 12 to 18 carbon atoms are also suitable, such as for example coco, palm, palm kernel or tallow fatty acid. PEG-40-monostearate, PEG-50-monostearate, PEG-100-monostearate, PEG-50-monooleate, PEG-100-monooleate, PEG-50-monolaurate and PEG-100-monolaurate are particularly preferred.

C₁₂-C₁₈ Alkanols or the C₁₂-C₁₈ carboxylic acids, each with 10-30 ethylene oxide units per molecule as well as mixtures of these substances, in particular Ceteth-10, Ceteth-12, Ceteth-20, Ceteth-30, Steareth-10, Steareth-12, Steareth-20, Steareth-30, Ceteareth-10, Ceteareth-12, Ceteareth-20, Ceteareth-30, Laureth-12 and Beheneth-20 are particularly preferably employed. Preferred sorbitol monoesters of optionally hydroxylated linear saturated and unsaturated C₁₂-C₃₀ carboxylic acids, ethoxylated with an average of 20-100 mol ethylene oxide per mol, are selected from Polysorbate-20, Polysorbate-40, Polysorbate-60 and Polysorbate-80.

Furthermore, C₈-C₂₂ alkyl mono and oligoglycosides are preferably employed. C₈-C₂₂ Alkyl mono and oligoglycosides represent known commercial surfactants and emulsifiers. Their manufacture results particularly from the reaction of glucose or oligosaccharides with primary alcohols including 8 to 22 carbon atoms. As far as the glycoside group is concerned, both monoglycosides, in which a cyclic sugar residue is glycosidically linked to the fatty alcohol, and also oligomeric glycosides, with a degree of oligomerization of up to 8, preferably 1-2, are suitable. In this context, the oligomerization degree is a statistical mean value based on the typical homolog distribution of such industrial products. Products that are available under the trade mark Plantacare® comprise a glucosidically linked C₈-C₁₆ alkyl group to an oligoglucoside residue, whose average degree of oligomerization is 1-2, in particular 1.2-1.4. Particularly preferred C₈-C₂₂ alkyl mono and oligoglycosides are selected from octyl glucoside, decyl glucoside, lauryl glucoside, palmityl glucoside, isostearyl glucoside, stearyl glucoside, arachidyl glucoside and behenyl glucoside as well as mixtures thereof. The acyl glucamides derived from glucamine are also suitable non-ionic oil in water emulsifiers.

Ethoxylated sterols, in particular ethoxylated soya sterols, also illustrate inventively suitable oil in water emulsifiers. The degree of ethoxylation must be greater than 5, preferably at least 10, in order to exhibit an HLB value greater the 7. Suitable commercial products are e.g. PEG-10 Soy Sterol, PEG-16 Soy Sterol and PEG-25 Soy Sterol.

Furthermore, partial esters of polyglycerins with 2 to 10 glycerin units and esterified with 1 to 4 saturated or unsaturated, linear or branched, optionally hydroxylated C₈-C₃₀ fatty acid residues are preferably employed, in so far as they have an HLB value of more than 7. Diglycerin monocaprylate, diglycerin monocaprate, diglycerin monolaurate, triglycerin monocaprylate, triglycerin monocaprate, triglycerin monolaurate, tetraglycerin monocaprylate, tetraglycerin monocaprate, tetraglycerin monolaurate, pentaglycerin monocaprylate, pentaglycerin monocaprate, pentaglycerin monolaurate, hexaglycerin monocaprylate, hexaglycerin monocaprate, hexaglycerin monolaurate, hexaglycerin monomyristate, hexaglycerin monostearate, decaglycerin monocaprylate, decaglycerin monocaprate, decaglycerin monolaurate, decaglycerin monomyristate, decaglycerin monoisostearate, decaglycerin monostearate, decaglycerin monooleate, decaglycerin monohydroxystearate, decaglycerin dicaprylate, decaglycerin dicaprate, decaglycerin dilaurate, decaglycerin dimyristate, decaglycerin diisostearate, decaglycerin distearate, decaglycerin dioleate, decaglycerin dihydroxystearate, decaglycerin tricaprylate, decaglycerin tricaprate, decaglycerin trilaurate, decaglycerin trimyristate, decaglycerin triisostearate, decaglycerin tristearate, decaglycerin trioleate and decaglycerin trihydroxystearate are particularly preferred.

An inventively preferred embodiment as a deodorant spray is characterized in that it comprises:
a) 0-7 wt % free water,
b) in total 1-95 wt %, preferably 5-90 wt %, particularly preferably 30-75 wt %, exceptionally preferably 50-60 wt %, of at least one cosmetic oil that is liquid under normal conditions,
c) an oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention,
d) in addition to the abovementioned components one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %, wherein the quantities refer to the weight of the composition, without taking into account optionally present propellant.

An inventively preferred embodiment as an antiperspirant spray is characterized in that it comprises:
a) 0-7 wt % free water,
b) in total 30-95 wt %, preferably 40-90 wt %, particularly preferably 50-85 wt %, exceptionally preferably 54-80 wt %, more preferably 55-75 wt %, likewise very preferably 60-70 wt %, of at least one cosmetic oil that is liquid under normal conditions,
c) an oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention,
d) in addition to the abovementioned components, one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %,
e) at least one antiperspirant active substance, selected from aluminum salts, in a total amount of 3-40 wt %, preferably 5-35 wt % and particularly preferably 10-25 wt %, relative to the total weight of the active substance anhydrous (USP) in the total composition,
   wherein the indications on quantities refer to the weight of the composition, without taking into account optionally present propellant.

An inventively preferred embodiment as a deodorant wax stick is characterized in that it comprises:
a) 0-7 wt % free water,
b) in total 30-70 wt % of at least one cosmetic oil that is liquid under normal conditions,
c) an oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention,
d) in addition to the abovementioned components, one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %,
e) 15-32 wt % of a fatty component that is solid under normal conditions of which more than 65 wt %, preferably more than 70 wt %, particularly preferably more than 80 wt %, each relative to the total content of fatty components that are solid under normal conditions, of C₁₆-C₃₀ fatty alcohols, preferably selected from the group consisting of stearyl alcohol, cetyl alcohol and mixtures thereof, wherein, 0.1-3 wt %, relative to the whole stick, of arachidyl alcohol and/or behenyl alcohol and/or at least one C₂₄-C₃₀ fatty alcohol is/are particularly preferably, additionally comprised in addition to stearyl alcohol and/or cetyl alcohol,
f) in addition 0.5-5 wt %, relative to the whole stick, of at least one fatty component with a melting point of 75-120° C. and 0.5-8 wt %, relative to the whole stick, of at least one fatty component with a melting point of 25-45° C.,
g) in addition 0.5-30 wt %, preferably 1-25 wt %, particularly preferably 5-20 wt %, exceptionally preferably 10-15 wt %, of at least one filler that is particularly preferably selected from talc, cellulose powder, starch and starch derivatives,
   wherein the indications on quantities refer to the weight of the composition.

An inventively preferred embodiment as a deodorant wax stick is characterized in that it comprises:
a) 0-7 wt % free water,
b) in total 30-70 wt % of at least one cosmetic oil that is liquid under normal conditions,
c) an oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention,
d) in addition to the abovementioned components, one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %,
e) 15-32 wt % of a fatty component that is solid under normal conditions of which more than 65 wt %, preferably more than 70 wt %, particularly preferably more than 80 wt %, each relative to the total content of fatty components that are solid under normal conditions, of C₁₆-C₃₀ fatty alcohols, preferably selected from the group consisting of stearyl alcohol, cetyl alcohol and mixtures thereof, wherein, 0.1-3 wt %, relative to the whole stick, of arachidyl alcohol and/or behenyl alcohol and/or at least one C₂₄-C₃₀ fatty alcohol is/are particularly preferably, additionally comprised in addition to stearyl alcohol and/or cetyl alcohol,
f) in addition 0.5-5 wt %, relative to the whole stick, of at least one fatty component with a melting point of 75-120° C. and 0.5-8 wt %, relative to the whole stick, of at least one fatty component with a melting point of 25-50° C.,
g) in addition 0.5-30 wt %, preferably 1-25 wt %, particularly preferably 5-20 wt %, exceptionally preferably 10-15 wt %, of at least one filler that is particularly preferably selected from talc, cellulose powder, starch and starch derivatives,
h) as well as 0.1-10 wt %, preferably 1-5 wt %, particularly preferably 2-4 wt %, each relative to the whole stick, of at least one oil-in-water emulsifier,
   wherein the indications on quantities refer to the weight of the composition.

An inventively preferred embodiment as an antiperspirant wax stick is characterized in that it comprises:
a) 0-7 wt % free water,
b) in total 30-70 wt % of at least one cosmetic oil that is liquid under normal conditions,
c) an oily extract from the leaves of Rumex acetosa in a total amount of 0.0001-5 wt %, preferably 0.001-2 wt %, particularly preferably. 0.01-1.5 wt %, exceptionally preferably 0.03-1 wt %, additionally preferably 0.1-0.5 wt %, each relative to the total weight of dry extract in the composition according to the invention,
d) in addition to the abovementioned components, one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %,
e) 15-32 wt % of a fatty component that is solid under normal conditions of which more than 65 wt %, preferably more than 70 wt %, particularly preferably more than 80 wt %, each relative to the total content of fatty components that are solid under normal conditions, of C₁₆-C₃₀ fatty alcohols, preferably selected from the group consisting of stearyl alcohol, cetyl alcohol and mixtures thereof, wherein, 0.1-3 wt %, relative to the whole stick, of arachidyl alcohol and/or behenyl alcohol and/or at least one C₂₄-C₃₀ fatty alcohol is/are particularly preferably, additionally comprised in addition to stearyl alcohol and/or cetyl alcohol,
f) in addition 0.5-5 wt %, relative to the whole stick, of at least one fatty component with a melting point of 75-120° C. and 0.5-8 wt %, relative to the whole stick, of at least one fatty component with a melting point of 25-50° C.,
g) in addition 0.5-30 wt %, preferably 1-25 wt %, particularly preferably 5-20 wt %, exceptionally preferably 10-15 wt %, of at least one filler that is particularly preferably selected from talc, cellulose powder, starch and starch derivatives,
h) at least one antiperspirant active substance, selected from aluminum salts, in a total amount of 3-27 wt %, preferably 5-22 wt % and particularly preferably 10-20 wt %, relative to the total weight of the active substance anhydrous (USP) in the total composition,
   wherein the indications on quantities refer to the weight of the composition.

Another inventively preferred embodiment as an antiperspirant wax stick is characterized in that it comprises:
a) 0-7 wt % free water,
b) in total 30-70 wt % of at least one cosmetic oil that is liquid under normal conditions,
c) an oily extract from the leaves of Rumex acetosa in a total amount of 0.0001-5 wt %, preferably 0.001-2 wt %, particularly preferably 0.01-1.5 wt %, exceptionally preferably 0.03-1 wt %, additionally preferably 0.1-0.5 wt %, each relative to the total weight of dry extract in the composition according to the invention,
d) in addition to the abovementioned components, one or more cooling agents in a total amount of 0.01-5 wt %, preferably in a total amount of 0.05-2 wt %, particularly preferably in a total amount of 0.1 to 1 wt %, exceptionally preferably in a total amount of 0.2-0.6 wt %,
e) 15-32 wt % of a fatty component that is solid under normal conditions of which more than 65 wt %, preferably more than 70 wt %, particularly preferably more than 80 wt %, each relative to the total content of fatty components that are solid under normal conditions, of C₁₆-C₃₀ fatty alcohols, preferably selected from the group consisting of stearyl alcohol, cetyl alcohol and mixtures thereof, wherein, 0.1-3 wt %, relative to the whole stick, of arachidyl alcohol and/or behenyl alcohol and/or at least one C₂₄-C₃₀ fatty alcohol is/are particularly preferably, additionally comprised in addition to stearyl alcohol and/or cetyl alcohol,
f) in addition 0.5-5 wt %, relative to the whole stick, of at least one fatty component with a melting point of 75-120° C. and 0.5-8 wt %, relative to the whole stick, of at least one fatty component with a melting point of 25-50° C.,
g) in addition 0.5-30 wt %, preferably 1-25 wt %, particularly preferably 5-20 wt %, exceptionally preferably 10-15 wt %, of at least one filler that is particularly preferably selected from talc, cellulose powder, starch and starch derivatives,
h) at least one antiperspirant active substance, selected from aluminum salts, in a total amount of 3-27 wt %, preferably 5-22 wt % and particularly preferably 10-20 wt %, relative to the total weight of the active substance anhydrous (USP) in the total composition and
i) 0.1-10 wt %, preferably 1-5 wt %, particularly preferably 2-4 wt %, each relative to the whole stick, of at least one oil-in-water emulsifier,
   wherein the indications on quantities refer to the weight of the composition.

Preferred inventive compositions comprise at least one antiperspirant active substance. Preferred antiperspirant active substances are selected from aluminum salts, preferably from the water-soluble astringent inorganic and organic salts of aluminum and aluminum-zirconium mixtures. Alumosilicates and zeolites are not inventively counted in the antiperspirant active substances.

According to the invention, water-solubility is understood to mean a solubility of at least 5 wt. % at 20° C., i.e. that quantities of at least 5 g of the antiperspirant active substance are soluble in 95 g water at 20° C.

Particularly preferred antiperspirant active substances are selected from aluminum chlorohydrate, in particular aluminum chlorohydrate with the general Formula [AI₂(OH)₅CI.1-6H₂O]ₙ, preferably [AI₂(OH)₅CI.2-3H₂O]ₙ, which can exist in the non-activated or in the activated (depolymerized) form, as well as aluminum chlorohydrate with the general Formula [AI₂(OH)₄CI₂.1-6H₂O]ₙ, preferably [AI₂(OH)₄CI₂.2-3H₂O]ₙ, which can exist in the non-activated or in the activated (depolymerized) form.

The production of preferred antiperspirant active substances is disclosed for example in U.S. Pat. No. 3,887,692, U.S. Pat. No. 3,904,741, U.S. Pat. No. 4,359,456, GB 2048229 and GB 1347950.

Aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex-PG or aluminum sesquichlorohydrex-PEG, aluminum PG-dichlorohydrex or aluminum PEG dichlorohydrex, aluminum hydroxide, additionally selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum zirconium chlorohydrate glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAI(SO₄)₂.12H₂O, alum), aluminum undecylenoyl collagen amino acid, sodium aluminum lactate+aluminum sulfate, sodium aluminum chlorohydroxy lactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxy allantoinate, sodium aluminum chlorohydroxy lactate, zinc chloride, zinc sulfocarbolate, zinc sulfate, zirconyl oxy halides, in particular zirconyl oxychlorides, zirconyl hydroxyhalides, in particular zirconyl hydroxychlorides (zirconium chlorohydrate) are also preferred.

Inventively particularly preferred antiperspirant active substances are selected from the “activated” aluminum and aluminum zirconium salts, which are also called enhanced activity antiperspirant active substances. These types of active substances are known from the prior art and are also commercially available. Their manufacture is disclosed in GB 2048229, U.S. Pat. No. 4,775,528 and U.S. Pat. No. 6,010,688, for example. Activated aluminum and aluminum zirconium salts are generally produced by heat treating a relatively dilute solution of the salt (e.g. about 10 wt % salt) in order to increase its HPLC peak 4 to peak 3 surface ratio. The activated salt can then be dried to a powder, in particular spray dried. In addition to spray drying, roll-mill drying for example is also suitable.

Activated aluminum and aluminum zirconium salts typically have an HPLC peak 4 to peak 3 surface ratio of at least 0.4, preferably at least 0.7, particularly preferably at least 0.9, wherein at least 70% of the aluminum is assigned to these peaks.

Activated aluminum and aluminum zirconium salts do not necessarily have to be employed as the spray dried powder. Likewise, non-aqueous solutions or solubilisates of an activated antiperspirant aluminum or aluminum zirconium salt, for example according to U.S. Pat. No. 6,010,688, are inventively preferred antiperspirant active substances. The addition of an effective amount of a polyhydric alcohol that has 3 to 6 carbon atoms and 3 to 6 hydroxy groups, preferably propylene glycol, sorbitol and pentaerythritol, stabilizes these salts against the loss of the activation and against the rapid reduction of the HPLC peak 4:peak 3 surface ratio of the salt. Exemplary preferred compositions comprise in weight percent (USP): 18-45 wt % of an activated aluminum or aluminum zirconium salt, 55-82 wt % of at least one anhydrous polyhydric alcohol including 3 to 6 carbon atoms and 3 to 6 hydroxy groups, preferably propylene glycol, butylene glycol, diethylene glycol, dipropylene glycol, glycerin, sorbitol and pentaerythritol, particularly preferably propylene glycol.

Complexes of the activated antiperspirant aluminum or aluminum zirconium salts with a polyhydric alcohol which comprise 20-50 wt %, particularly preferably 20-42 wt %, of the activated antiperspirant aluminum or aluminum zirconium salt and 2-16 wt % of molecularly bound water are also particularly preferred, wherein the remainder that makes up 100 wt % is at least one polyhydric alcohol including 3 to 6 carbon atoms and 3 to 6 hydroxy groups. Propylene glycol, propylene glycol/sorbitol mixtures and propylene glycol/pentaerythritol mixtures are preferred alcohols of this type. These kinds of inventively preferred complexes of an activated antiperspirant aluminum or aluminum zirconium salt with a polyhydric alcohol are disclosed, for example, in U.S. Pat. No. 5,643,558 and U.S. Pat. No. 6,245,325.

Other preferred antiperspirant active substances are basic calcium aluminum salts, such as are disclosed, for example, in U.S. Pat. No. 2,571,030. These salts are manufactured by treating calcium carbonate with aluminum chlorohydroxide or aluminum chloride and aluminum powder or by adding calcium chloride dihydrate to aluminum chlorohydroxide.

Other preferred antiperspirant active substances are aluminum zirconium complexes, such as those for example disclosed in U.S. Pat. No. 4,017,599, which are buffered with amino acid salts, in particular with alkali metal and alkaline earth metal glycinates.

Other preferred antiperspirant active substances are activated aluminum or aluminum zirconium salts, such as those for example disclosed in U.S. Pat. No. 6,245,325 or U.S. Pat. No. 6,042,816, comprising 5-78 wt % (USP) of an activated aluminum or aluminum zirconium salt, an amino acid or hydroxyalkanoic acid in an amount such that an (amino acid or hydroxyalkanoic acid) to (Al+Zr) weight ratio of 2:1-1:20 and preferably 1:1-1:10 is obtained, as well as a water-soluble calcium salt in an amount such that a Ca:(A+Zr) weight ratio of 1:1-1:28 and preferably 1:2-1:25 is obtained. Particularly preferred solid activated antiperspirant salt compositions, for example according to U.S. Pat. No. 6,245,325 or U.S. Pat. No. 6,042,816, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble calcium salt such that the Ca:(Al+Zr) weight ratio is 1:1-1:28, preferably 1:2-1:25, and as much amino acid, such that the amino acid to (Al+Zr) weight ratio is 2:1-1:20, preferably 1:1-1:10.

Other particularly preferred solid activated antiperspirant salt compositions, for example according to U.S. Pat. No. 6,245,325 or U.S. Pat. No. 6,042,816, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble calcium salt such that the Ca:(Al+Zr) weight ratio is 1:1-1:28, preferably 1:2-1:25, and as much glycine, such that the glycine to (Al+Zr) weight ratio is 2:1-1:20, preferably 1:1-1:10.

Other particularly preferred solid activated antiperspirant salt compositions, for example according to U.S. Pat. No. 6,245,325 or U.S. Pat. No. 6,042,816, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble calcium salt such that the Ca:(Al+Zr) weight ratio is 1:1-1:28, preferably 1:2-1:25, and as much hydroxyalkanoic acid, such that the hydroxyalkanoic acid to (Al+Zr) weight ratio is 2:1-1:20, preferably 1:1-1:10.

Preferred water-soluble calcium salts for the stabilization of the antiperspirant salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, as well as mixtures thereof.

Preferred amino acids for the stabilization of the antiperspirant salts are selected from glycine, alanine, leucine, isoleucine, β-alanine, valine, cysteine, serine, tryptophan, phenylalanine, methionine, β-amino-n-butyric acid and γ-amino-n-butyric acid and the salts thereof, each in the d-form, der l-form and the dl-form; glycine is particularly preferred.

Preferred hydroxyalkanoic acids for the stabilization of the antiperspirant salts are selected from glycolic acid and lactic acid.

Other preferred antiperspirant active substances are activated aluminum or aluminum zirconium salts, such as those for example disclosed in U.S. Pat. No. 6,902,723, comprising 5-78 wt % (USP) of an activated antiperspirant aluminum or aluminum zirconium salt, an amino acid or hydroxyalkanoic acid in an amount such that an (amino acid or hydroxyalkanoic acid) to (Al+Zr) weight ratio of 2:1-1:20 and preferably 1:1-1:10 is obtained, as well as a water-soluble strontium salt in an amount such that a Sr:(Al+Zr) weight ratio of 1:1-1:28 and preferably 1:2-1:25 is obtained.

Particularly preferred solid activated antiperspirant salt compositions, for example according to U.S. Pat. No. 6,902,723, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble strontium salt such that the Sr:(Al+Zr) weight ratio is 1:1-1:28, preferably 1:2-1:25, and as much amino acid, such that the amino acid to (Al+Zr) weight ratio is 2:1-1:20, preferably 1:1-1:10.

Other particularly preferred solid activated antiperspirant salt compositions, for example according to U.S. Pat. No. 6,902,723, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble strontium salt such that the Sr:(Al+Zr) weight ratio is 1:1-1:28, preferably 1:2-1:25, and as much glycine, such that the glycine to (Al+Zr) weight ratio is 2:1-1:20, preferably 1:1-1:10.

Other particularly preferred solid activated antiperspirant salt compositions, for example according to U.S. Pat. No. 6,902,723, comprise 48-78 wt % (USP), preferably 66-75 wt % of an activated aluminum or aluminum zirconium salt and 1-16 wt %, preferably 4-13 wt % of molecularly bound water (water of hydration), additionally as much water-soluble strontium salt such that the Sr:(Al+Zr) weight ratio is 1:1-1:28, preferably 1:2-1:25, and as much hydroxyalkanoic acid, such that the hydroxyalkanoic acid to (Al+Zr) weight ratio is 2:1-1:20, preferably 1:1-1:10.

Further preferred activated aluminum salts are those with the general Formula AI₂(OH)₆₋ₐXa, wherein X is Cl, Br, I or NO₃ and “a” is 0.3 to 5, preferably 0.8 to 2.5 and particularly preferably 1 to 2, such that the mole ratio of AI to X is in the range 0.9:1 to 2.1:1, as are disclosed for example in U.S. Pat. No. 6,074,632. With these salts there is generally some associatively bound water of hydration, typically 1 to 6 moles water per mole salt. Aluminum chlorohydrate is particularly preferred (i.e. X is Cl in the above formula) and specifically 5/6-basic aluminum chlorohydrate, in which “a” is 1, such that the molar ratio of aluminum to chlorine is 1.9:1 to 2.1:1.

Preferred activated aluminum zirconium salts are those that illustrate mixtures or complexes of the above described aluminum salts with zirconium salts of the Formula ZrO(OH)_{2-b}Y_{b}, in which Y is Cl, Br, I, NO₃ or SO₄, b is a rational number from 0.8 to 2 and p is the valency of Y, as are disclosed for example in U.S. Pat. No. 6,074,632. As a rule, the zirconium salts likewise have some associatively bound water of hydration, typically in amounts of 1 to 7 moles water per mole salt. The zirconium salt is preferably zirconyl hydroxychloride of the Formula ZrO(OH)_{2-b}Cl_{b}, in which b is a rational number from 0.8 to 2, preferably 1.0 to 1.9. Preferred aluminum zirconium salts have an Al:Zr molar ratio of 2 to 10 and a metal:(X+Y) ratio of 0.73 to 2.1, preferably 0.9 to 1.5. A particularly preferred salt is aluminum zirconium chlorohydrate (i.e., X and Y are Cl), which has an Al:Zr ratio of 2 to 10 and a molar metal:Cl ratio of 0.9 to 2.1. The term aluminum zirconium chlorohydrate includes the tri, tetra, penta and octachlorohydrate forms.

Inventively preferred zirconium salts have the general Formula ZrO(OH)₂₋ₐClₐ^{▪}x H₂O with a=1.5-1.87; x=1-7, wherein a and x are rational numbers. These zirconium salts are disclosed for example in the Belgian document BE 825146.

Other preferred antiperspirant active substances are disclosed in U.S. Pat. No. 6,663,854 and US 20040009133.

The antiperspirant active substances can be in both solubilized as well as in undissolved, suspended form.

In so far as the antiperspirant active substances are suspended in a water-immiscible carrier, then for reasons of product stability the particles of the active substance preferably exhibit a number average particle size of 0.1-200 μm, preferably 1-50 μm, particularly preferably 3-20 μm and exceptionally preferably 5-10 μm.

Preferred aluminum salts and aluminum zirconium salts have a molar metal to chloride ratio of 0.9 to 1.3, preferably 0.9 to 1.1, particularly preferably 0.9 to 1.0.

Preferred aluminum zirconium chlorohydrates generally have the empirical Formula AlₙZr(OH)_{[3n+4-m(n+1)]}(CI)_{[m(n+1)]} with n=2.0-10.0, preferably 3.0-8.0, m=0.77-1.11 (corresponding to a molar metal (AI+Zr) to chloride ratio of 1.3-0.9), preferably m=0.91-1.11 (corresponding to M:CI=1.1-0.9), and particularly preferably m=1.00-1.11 (corresponding to M:CI=1.0-0.9), additionally very preferably m=1.02-1.11 (corresponding to M:CI=0.98-0.9) and very preferably m=1.04-1.11 (corresponding to M:CI=0.96-0.9).

With these salts there is generally some associatively bound water of hydration, typically 1 to 6 moles water per mole salt, corresponding to 1-16 wt %, preferably 4-13 wt % water of hydration.

The preferred aluminum zirconium chlorohydrates are usually associated with an amino acid in order to prevent polymerization of the zirconium species during production. Preferred stabilizing amino acids are selected from glycine, alanine, leucine, isoleucine, β-alanine, valine, cysteine, serine, tryptophan, phenylalanine, methionine, β-amino-n-butyric acid and γ-amino-n-butyric acid and the salts thereof, each in the d-form, the l-form and the dl-form; glycine is particularly preferred. The amino acid is comprised in an amount of 1-3 mol, preferably 1.3-1.8 mol, each per mol zirconium in the salt.

Preferred antiperspirant salts are aluminum zirconium tetrachlorohydrate (AI:Zr=2-6; M:CI=0.9-1.3), in particular salts with a molar metal to chloride ratio of 0.9-1.1, preferably 0.9-1.0.

Aluminum zirconium chlorohydrate glycine salts that are stabilized with betaine ((CH₃)₃N⁺—CH₂—COO⁻) are additionally inventively preferred. Particularly preferred corresponding compounds have a molar total (betaine+glycine)/Zr ratio of (0.1-3.0):1, preferably (0.7-1.5):1 and a molar ratio of betaine to glycine of at least 0.001:1. Suitable compounds are disclosed for example in U.S. Pat. No. 7,105,691.

In a particularly preferred embodiment according to the invention, an activated salt is comprised as the particularly effective antiperspirant salt, in particular one with a high HPLC peak 5 aluminum content, in particular with a peak 5 surface area of at least 33%, particularly preferably at least 45%, relative to the total surface under the peaks 2-5, measured with HPLC of a 10 wt % conc. aqueous solution of the active substance under conditions, in which the aluminum species are resolved into at least 4 sequential peaks (with peaks 2-5 labeled). Preferred aluminum zirconium salts with a high HPLC peak 5-aluminum content (also labelled as “E⁵ AZCH”) are disclosed for example in U.S. Pat. No. 6,436,381 and U.S. Pat. No. 6,649,152.

Moreover, such activated “E⁵ AZCH” salts are preferred, whose HPLC peak 4 to peak 3 surface area ratio is at least 0.4, preferably at least 0.7, particularly preferably at least 0.9.

Other particularly preferred antiperspirant active substances are those aluminum zirconium salts with a high HPLC peak 5 aluminum content which are additionally stabilized with a water-soluble strontium salt and/or with a water-soluble calcium salt. Suitable salts are disclosed for example in U.S. Pat. No. 6,923,952.

The anti-perspirant active substances can be used as non-aqueous solutions or as glycolic solubilisates.

Particularly preferred inventive compositions are characterized in that they comprise at least one antiperspirant active substance in a total amount of 3-27 wt %, preferably 5-22 wt % and particularly preferably 10-20 wt %, relative to the total weight of the active substance (USP), free of water of crystallization, in the total composition.

In a particularly preferred embodiment, the composition comprises an astringent aluminum salt, especially aluminum chlorohydrate, which is commercially available, for example, in powder form as Micro Dry® Ultrafine from Reheis, as Microdry 323 from Summit, as Chlorhydrol® as well as in activated form as Reach® 501 from Reheis. An aluminum sesquichlorohydrate is offered by Reheis under the name Reach® 301, which is also particularly preferred. Activated aluminum chlorohydrates that are available under the designations Reach® 101 and Reach® 103, AACH-7171 from Reheis or Summit are also particularly preferred. The use of aluminum zirconium tetrachlorohydrex glycine complexes that are commercialized for example by Reheis under the name Rezal® 36 GP by Reheis or AZG-364 or 369 by Summit, in activated quality, as Reach® 908, as a powder, can also be inventively particularly preferred. Aluminum zirconium pentachlorohydex glycine complexes (AAZG-3108 or AAZG-3110 from Summit) can also be employed.

The antiperspirant active substances are in undissolved, suspension form.

In so far as the antiperspirant active substances are suspended in a water-immiscible carrier, then for reasons of product stability the particles of the active substance preferably exhibit a number average particle size of 0.1-200 μm, preferably 1-50 μm, particularly preferably 3-20 μm and exceedingly preferably 5-10 μm. Preferred active substance particles have a volumetric mean particle size of 0.2-220 μm, preferably 3-60 μm, particularly preferably 4-25 μm, further preferably 5-20 μm and extremely preferably 10-15.5 μm.

Preferred aluminum salts have a molar metal to chloride ratio of 1.9 to 2.1, or for sequichlorohydrates from 1.5:1 to 1.8:1.

Particularly preferred inventive compositions are characterized in that they comprise at least one antiperspirant active substance in a total amount of 5-40 wt %, preferably 10-35 wt %, particularly preferably 11-28 wt % and extremely preferably 12-20 wt %, relative to the total weight of the active substance (USP), free of water of crystallization, in the propellant-free total composition.

In a particularly preferred embodiment, the composition comprises an astringent aluminum salt, in particular aluminum chlorohydrate, particularly preferably aluminum chlorohydrate with an active substance (USP) free of water of crystallization of 72-88 wt %, relative to the raw material as is. Preferred unactivated aluminum chlorohydrates are marketed for example in powder form as Micro Dry®, Micro Dry® Ultrafine or Micro Dry®-323 from Summit/Reheis, as Chlorhydrol® (powder) as well as in activated form as Reach® 101, Reach® 103, Reach® 501 from Reheis/Summit or AACH-7171 from Summit. An aluminum sesquichlorohydrate is offered under the name Reach® 301 by Reheis and is also particularly preferred.

Furthermore, it was found that the release of the antiperspirant active substance from an inventive antiperspirant composition can be improved if at least one organosiloxane-oxyalkylene copolymer is comprised.

Inventively preferred compositions are characterized in that the organosiloxane-oxyalkylene copolymer is selected from compounds of the general structural Formula (II),

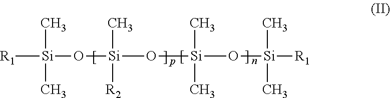

wherein
the R₁ groups independently of one another represent a linear or branched C₁-C₃₀ alkyl group or an optionally substituted phenyl group, preferably a methyl group,
the R² groups, independently of one another, represent the groups —C_{c}—H_{2c}—O—(C₂H₄O—)ₐ(C₃H₆O—)_{b}R⁵ or —C_{c}H_{2c}—O—(C₂H₄O—)ₐR⁵ or —CH₂—CH(CH₃)—CH₂—O—(C₂H₄O—)ₐ(C₃H₆O—)_{b}R⁵,
n represents a number from 0-500, preferably 20-400, particularly preferably 50-300,
p represents a number from 1-50, preferably 10-40, particularly preferably 20-30.

Those organosiloxane-oxyalkylene copolymers of the above illustrated general structural Formula (II), in which the R¹ groups independently of each other represent a linear or branched C₁-C₃₀ alkyl group, preferably a linear or branched C₁-C₁₆ alkyl group, particularly preferably a linear or branched C₁-C₄ alkyl group, exceedingly preferably a methyl group, are preferred for the inventive teaching. Particularly preferred linear or branched C₁-C₄ alkyl groups are selected from methyl, ethyl, 1-methylethyl, n-propyl, n-butyl, tert-butyl and 2-methylpropyl.

Other preferred compositions according to the invention comprise at least one organosiloxane-oxyalkylene copolymer of the above illustrated general structural Formula (II) which has an HLB value in the range 8-20, preferably 10-18, particularly preferably 11-16.

Other preferred compositions according to the invention comprise at least one organosiloxane-oxyalkylene copolymer of the above illustrated general structural Formula (II), in which the R¹ groups independently of each other represent a linear or branched C₁-C₃₀ alkyl group, preferably a linear or branched C₁-C₁₆ alkyl group, particularly preferably a linear or branched C₁-C₄ alkyl group, in particular methyl, ethyl, 1-methylethyl, n-propyl, n-butyl, tert-butyl and 2-methylpropyl, extremely preferably a methyl group, and have an HLB value in the range 8-20, preferably 10-18, particularly preferably 11-16.

Inventively particularly preferred compositions comprise at least one organosiloxane-oxyalkylene copolymer selected from compounds of the general structural Formula (II) with an HLB value in the range 8-20, preferably 10-18, particularly preferably 11-16, and with
R¹=methyl group, R²=—C_{c}H_{2c}—O—(C₂H₄O—)ₐR⁵,
R⁵=a hydrogen atom or a methyl group,
n=0, p=1, a=5-20, preferably 7-15, particularly preferably 8-11, b=0, c=3.

Other inventively particularly preferred organosiloxane-oxyalkylene copolymers are selected from linear polysiloxane-polyoxyalkylene block copolymers, in particular from linear polysiloxane-polyoxyethylene-polyoxypropylene block copolymers. A linear polysiloxane-polyoxyethylene-polyoxypropylene block copolymer with the INCI name PEG/PPG/24 Dimethicone is inventively exceptionally preferred. A linear polysiloxane-polyoxyethylene-polyoxypropylene block copolymer of this type is available for example under the trade name Mirasil DMCO (INCI: PEG/PPG-22/24 Dimethicone) from the Bluestar Company (Rhodia). Another preferred linear polysiloxane-polyoxyethylene-polyoxypropylene block copolymer of this type has the INCI name PEG/PPG-10/2 Dimethicone. It is available for example under the trade name Mirasil DMCP 93 (INCI: PEG/PPG-10/2 Dimethicone) from Bluestar (Rhodia).

Other inventively particularly preferred compositions comprise at least one organosiloxane-oxyalkylene copolymer, selected from PEG/PPG-18/18 Dimethicone, PEG-12 Dimethicone, PEG/PPG-22/24 Dimethicone, PEG/PPG-17/18 Dimethicone, PEG/PPG-20/6 Dimethicone, PEG/PPG-14/4 Dimethicone, Bis-PEG/PPG-16/16 PEG/PPG-16/16 Dimethicone as well as mixtures thereof.

Other preferred inventive compositions comprise at least one of the previously named organosiloxane-oxyalkylene copolymers in a total amount of 0.01-5 wt %, preferably 0.1-3 wt %, particularly preferably 0.5 to 2 wt %, exceedingly preferably 0.7-1.5 wt %, wherein the quantities refer to the weight of the composition, without taking into account optionally present propellant.

The inventive compositions can also be presented as a massage fluid, for example as a massage oil, massage cream or massage lotion. An inventive massage fluid of this type produces a long lasting, pleasant cooling effect on the skin. A preferred inventive massage fluid additionally comprises peeling particles. The inventive compositions can also be presented as a foot-care agent, for example as a foot lotion. A preferred inventive foot-care agent additionally comprises a compound with keratolytic action which is selected from salicylic acid, sodium salicylate, fruit acids, in particular citric acid or glycolic acid, as well as mixtures thereof. The inventive compositions can also be presented as a shaving auxiliary, for example as a shaving cream. Preferred inventive compositions, in particular a preferred inventive shaving auxiliary, additionally comprises a compound with a skin calming action which is preferably selected from panthenol, allantoin, α-bisabolol, α-liponic acid, pentacyclic triterpene acids, in particular asiatic acid, 6-hydroxyasiatic acid (madecassic acid), betulinic acid and platanic acid, as well as the sugar esters of these pentacyclic triterpene acids, which can be extracted in particular from Centella asiatica (Indian pennywort) (see U.S. Pat. No. 7,402,669 B2), the physiologically acceptable salts of sterol sulfates, in particular sodium beta-sitosterol sulfate, amino dicarboxylic acids with a carbon chain length of 3-6 carbon atoms as well as their physiologically acceptable salts, preferably selected from amino malonic acid, amino succinic acid (=aspartic acid), amino glutaric acid and amino adipic acid as well as their physiologically acceptable salts such as potassium aspartate and magnesium aspartate, as well as any mixtures of these substances, preferably in a total amount of 0.001 to 5 wt %, particularly preferably 0.01 to 2 wt % and extremely preferably 0.1 to 1 wt %, each based on the total composition without taking account of optionally present propellant.

The inventive compositions can also be presented as oral care agents, for example as an oral rinse liquid, mouth spray or toothpaste. An inventive oral care agent of this type produces a pleasant long-lasting cooling effect on the mucous membrane of the oral cavity.

The inventive compositions that are applied as sprays are preferably made up according to the requirements of the desired spray application. The inventive compositions are in the form of a suspension, that is, the antiperspirant active substance and optional additional insoluble ingredients are suspended in a liquid carrier. A disperse system of this kind should be shaken prior to the application. In another inventively preferred embodiment, the inventive compositions are made up as suspension that can be sprayed with a propellant.

Preferred compositions according to the invention may, for example, be packaged in pump or squeeze dispensers, in particular in multi-chamber pump or squeeze dispensers. Such dispensers use air, in particular ambient air, as the propellant or deliver the inventive composition by means of pumps.

In a further preferred embodiment of the invention, the composition is applied by means of a compressed or liquefied propellant.

Unless otherwise stated, all quantities are given relative to the weight of the composition excluding propellant.

Packaging in a multi-chamber dispenser offers particular technical advantages.

The multi-chamber dispenser may also be used such that one chamber is filled with the composition according to the invention, while another chamber includes the compressed propellant. One such multi-chamber dispenser is for example a “bag-in-can” package.

The two chambers may, however, also be connected to one another in such a manner that the composition according to the invention is divided into two sub-compositions which may simultaneously be discharged from the package, for example from separate orifices or from a single orifice.

Further compositions which are preferred according to the invention are characterized in that they are packaged with at least one propellant in a suitable pressure includeer.

Propellants (propellant gases) which are preferred according to the invention are selected from propane, propene, n-butane, iso-butane, iso-butene, n-pentane, pentene, iso-pentane, iso-pentene, methane, ethane, dimethyl ether, nitrogen, air, oxygen, laughing gas, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-11-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethane, 1,1,1,3-tetrafluoroethane, heptafluoro-n-propane, perfluoroethane, monochlorodifluoromethane, 1,1-difluoroethane, and specifically both individually and in combination.

Propane, n-butane, iso-butane are particularly preferred and mixtures of these propellants are particularly preferred.

Hydrophilic propellant gases, such as for example carbon dioxide, may advantageously be used for the purposes of the present invention if a small proportion of hydrophilic gases is selected and a lipophilic propellant gas (for example propane/butane) is present in excess. Propane, n-butane, iso-butane and in particular mixtures of these propellant gases are particularly preferred.

The quantity of propellant preferably amounts to 20-95 wt %, particularly preferably to 30-90 wt % and extremely preferably to 60-86 wt %, and furthermore extremely preferably to 75-78 wt %, in each case relative to the total weight of the preparation consisting of the suspension according to the invention and the propellant.

Vessels made of metal (aluminum, tin plate, tin), safety or non-splintering plastic or glass come into consideration as the compressed gas includeer, for which the factors pressure and rupture strength, corrosion resistance, ease of filling as well as aesthetic considerations, handling, printability etc. play a role in the choice. Special protective inner lacquers ensure the corrosion resistance against the inventive suspension. An inventively preferred protective inner lacquer is an epoxy-phenol lacquer that is available inter alia under the name Hoba 7407 P. The valves that are used particularly preferably possess an internally lacquered valve disc, wherein lacquer and valve material are mutually compatible. If aluminum valves are used, their valve disc can be internally coated with e.g. Micoflex lacquer. If tin plate valves are inventively employed, their valve discs can be internally coated with e.g. PET (polyethylene terephthalate). Preferred inventive compositions comprise at least one encapsulated active substance. The active substances that can be advantageously encapsulated are in particular fragrances and/or cooling agents, but also other skin-care active substances, such as vitamins, antioxidants etc.

The capsule materials are preferably water-soluble polymers such as starch, physically and/or chemically modified starches, cellulose derivatives such as e.g. carboxymethyl cellulose, methyl cellulose, hydroxyethyl cellulose or hydroxypropyl methyl cellulose, carragheen, alginates, maltodextrins, dextrins, plant gums, pectins, xanthanes, polyvinyl acetate and polyvinyl alcohol, polyvinyl pyrrolidine, polyamides, polyesters and homopolymers and copolymers of monomers, selected from acrylic acid, methacrylic acid, maleic acid, fumaric acid, itaconic acid as well as the esters and the salts of these acids, as well as any mixtures of these polymers.

Preferred capsule materials are chemically modified starches, especially aluminum starch octenylsuccinate, e.g. the commercial product Dry Flo Plus from National Starch, or sodium starch octenylsuccinate, e.g. the commercial product Capsul 10 from National Starch, furthermore carboxymethyl celluloses, carboxymethyl cellulose, methyl cellulose, hydroxyethyl cellulose and hydroxypropyl methyl cellulose, ethyl cellulose, e.g. the commercial product Tylose H from Clariant, in addition carragheen, alginates and maltodextrins, as well as any mixtures of these polymers.

In another inventively preferred embodiment, the inventive compositions comprise 10-85 wt % ethanol, preferably 20-80 wt % ethanol, particularly preferably 30-75 wt % ethanol, each relative to the total propellant-free composition. In another inventively preferred embodiment, the inventive compositions comprise 0 to maximum 5 wt % ethanol, relative to the total propellant-free composition. For reasons of anticorrosion protection, ethanol contents of 0-3 wt % are preferred, with ethanol contents of 0-1 wt % being particularly preferred.

Furthermore, the inventive compositions can comprise additional deodorants. Antimicrobials, antibacterials or germ inhibiting substances, antioxidants or odor adsorbents (e.g. zinc ricinoleate) can be incorporated as the deodorants.

Suitable antimicrobials, antibacterials or germ inhibiting substances are in particular organohalogen compounds as well as organohalides, quaternary ammonium compounds, a series of plant extracts, colloidal, elementary silver, inorganic or organic silver salts, such as in particular silver citrate and silver dihydrogen citrate, and zinc compounds. Halogenated phenol derivatives are preferred, such as e.g. hexachlorophene or Irgasan DP 300 (Triclosan, 2,4,4-trichloro-2′-hydroxydiphenyl ether), 3,4,4′-trichlorocarbonilide, Chlorhexidine (1,1′-hexamethylene-bis-[5-(4-chlorophenyl)]-biguanide), Chlorhexidine gluconate, benzalkonium halides and cetylpyridinium chloride. Furthermore, sodium bicarbonate, sodium phenol sulfonate and zinc phenol sulfonate as well as e.g. the constituents of linden blossom oil can be incorporated. Weaker-acting antimicrobials that, however, have a specific action against Gram positive germs that are responsible for the decomposition of perspiration, can also be incorporated as the deodorant active substance. Benzyl alcohol can also be incorporated as the deodorant active substance. Further antibacterially active deodorants are lantibiotics, glycoglycero lipids, sphingo lipids (ceramides), sterols and other active substances that inhibit the adhesion of bacteria on the skin, e.g. glycosidases, lipases, proteases, carbohydrates, di and oligosaccharide fatty acid esters as well as alkylated mono and oligosaccharides. Preferred deodorant active substances are long chain diols, e.g. 1,2-alkane-(C₅-C₁₈) diols, in particular 1,2-hexane diol or 1,2-octane diol, glycerin mono (C₈-C₁₈) fatty acid esters or, particularly preferably, glycerin mono-(C₆-C₁₆) alkyl ethers, in particular 2-ethylhexyl glycerin ethers, which are highly compatible with skin and mucous membranes and are active against corynebacteria, as well as in addition phenoxyethanol, phenoxyisopropanol (3-phenoxy-propan-2-ol), aniseed alcohol, 2-methyl-5-phenyl-pentan-1-ol, 1,1-dimethyl-3-phenyl-propan-1-ol, benzyl alcohol, 2-phenylethan-1-ol, 3-phenylpropan-1-ol, 4-phenylbutan-1-ol, 5-phenyl-pentan-1-ol, 2-benzylheptan-1-ol, 2,2-dimethyl-3-phenylpropan-1-ol, 2,2-dimethyl-3-(3′-methyl-phenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethyl-propan-1-ol, 3-(3′,4′-dichlorophenyl)-2-ethylpropan-1-ol, 2-ethyl-3-(2′-methylphenyl)-propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3′,4′-dimethoxyphenyl)-2-ethylpropan-1-ol, 2-allyl-3-phenylpropan-1-ol and 2-n-pentyl-3-phenylpropan-1-ol.

Complexants can also support the deodorizing effect, in that they form stable complexes with the oxidatively catalytically active heavy metal ions (e.g. iron or copper), Suitable complexants are e.g. the salts of ethylenediaminetetraacetic acid or of nitrilotriacetic acid as well as the salts of 1-hydroxyethane-1,1-diphosphonic acid.

The inventive compositions are preferably packaged in customary aerosol cans. The cans can be made from tin plate or from aluminum. Furthermore, the cans can have an inside coating in order to keep the danger of corrosion as low as possible.

The cans are equipped with a suitable spraying head. Depending on the spraying head, the possible discharge rates, based on a fully filled can, are from 0.1 g/s to 2.0 g/s.

The following examples are intended to clarify the invention without limiting it in any way.

### Measuring the Cooling Effect

Inventive antiperspirant aerosols were produced with various cooling agents and evaluated by test persons. It was shown that the cooling agent L-menthol is employable only in very small quantities, namely in quantities below 0.1 wt %, relative to the weight of the gasified aerosol formulation, corresponding to a weight fraction of 0.66 wt %, relative to the weight of the formulation, without taking into account the propellant. With greater amounts, an unwanted feeling of dampness up to increased perspiration was observed. The skin compatibility in the armpit area was also worsened by menthol above 0.1 wt %, relative to the weight of the gasified aerosol formulation, corresponding to a weight fraction of 0.66 wt %, relative to the weight of the formulation, without taking into account the propellant. With the formulation A including 0.07 wt % L-menthol, relative to the gasified aerosol formulation, corresponding to 0.47 wt %, relative to the weight of the formulation, without taking into account the propellant, 57% of the tested persons observed a cooling effect with good skin compatibility. This average strength effect remained for an average of ca. 2 hours. The proportion of nonresponders, 47%, was too high, however, and could not be reduced by increasing the concentration of L-menthol without the appearance of the cited inacceptable side effects.

It was surprisingly found that an extract from Rumex acetosa leaves likewise has a cooling effect in the armpit, comparable with that of the L-menthol. The formulation B including 1.0 wt % of an extract of Rumex acetosa leaf in solution/suspension in isopropyl myristate with a dry substance weight of 0.5 wt %, relative to the weight of the raw material itself, was also found to be cooling by 57% of the test persons. This average strength effect remained for an average of ca. 3 hours. The amount of dry substance of the oily Rumex acetosa leaf extract here amounted to 0.033 wt %, relative to the weight of the inventive composition without propellant.

It was surprisingly found that with the combination of 0.07 wt % L-menthol with 0.005 wt % Rumex acetosa leaf dry extract (formulation C, all quantities relative to the weight of the propellant-including composition) all test persons perceived the cooling effect, without occasioning an increase in negative side effects. With the formulation C the strong cooling effect remained for 6 hours on average.

TABLE
Aerosol formulations with cooling effects (all quantities in wt %)

| | A | B | C |
|---|---|---|---|
| Cyclopentasiloxane (Dow Corning 245 Fluid) | 7.06 | 6.13 | 6.06 |
| Isopropyl myristate | 1.00 | 1.00 | 1.00 |
| Rumex acetosa leaf extract (0.5 wt % dry | — | 1.00 | 1.00 |
| extract in isopropyl myristate, | | | |
| extracted with isopropyl myristate | | | |
| L-Menthol | 0.07 | — | 0.07 |
| Aluminum chlorohydrate | 5.00 | 5.00 | 5.00 |
| Propylene carbonate | 0.13 | 0.13 | 0.13 |
| Disteardimonium Hectorite | 0.38 | 0.38 | 0.38 |
| Perfume | 1.36 | 1.36 | 1.36 |
| Propane | 12.0 | 12.0 | 12.0 |
| n-Butane | 71.50 | 71.50 | 71.50 |
| Isobutane | 1.50 | 1.50 | 1.50 |

Additional inventive exemplary compositions (Antiperspirant sprays)
(all quantities in wt %)

| | D | E | F | G | H | I | J |
|---|---|---|---|---|---|---|---|
| Cyclopentasiloxane (Dow Corning 245 | ad | ad | ad | ad | ad | ad | — |
| Fluid) | 100 | 100 | 100 | 100 | 100 | 100 | |
| Isopropyl myristate | 1.0 | 1.0 | 1.0 | 1.0 | 1.0 | — | — |
| Triethyl citrate | — | — | — | — | — | 1.0 | 1.5 |
| 2-Ethylhexyl palmitate | — | — | — | — | — | — | ad |
| | | | | | | | 100 |
| Rumex acetosa leaf extract (0.5 wt % dry | 1.0 | 1.0 | 1.0 | 1.0 | 1.0 | 1.0 | 1.0 |
| extract in isopropyl myristate, | | | | | | | |
| extracted with isopropyl myristate | | | | | | | |
| L-Menthol | 0.07 | — | — | — | — | — | — |
| L-Menthyl lactate | — | 0.07 | 0.1 | — | — | — | 0.1 |
| N-2,3-Trimethyl-2-isopropylbutamide | — | — | 0.1 | — | — | — | — |
| N-Ethyl-p-menthane-3-carboxamide | — | — | 0.1 | — | — | — | — |
| Isopulegol | — | — | — | 0.2 | — | — | — |
| Cyanomethylphenyl menthane | — | — | — | — | 0.3 | 0.2 | 0.1 |
| carboxamide | | | | | | | |
| Aluminum chlorohydrate | 5.0 | 5.0 | 5.0 | 5.0 | 5.0 | 5.0 | 5.0 |
| Propylene carbonate | 0.13 | 0.13 | 0.13 | 0.13 | 0.13 | 0.13 | 0.13 |
| Disteardimonium Hectorite | 0.38 | 0.38 | 0.38 | 0.38 | 0.38 | 0.38 | 0.38 |
| Perfume | 1.36 | 1.36 | 1.36 | 1.36 | 1.36 | 1.36 | 1.36 |
| Propane | 12.0 | 12.0 | 12.0 | 12.0 | 12.0 | 12.0 | 12.0 |
| n-Butane | 71.1 | 71.1 | 71.1 | 71.1 | 71.1 | 71.1 | 71.1 |
| Isobutane | 1.5 | 1.5 | 1.5 | 1.5 | 1.5 | 1.5 | 1.5 |

Antiperspirant wax stick (all quantities in wt %)

| | K | L | M |
|---|---|---|---|
| Hexyldecanol | 10.00 | — | — |
| PPG-14 butyl ether | 6.00 | 15.00 | 13.00 |
| Hydrogenated castor oil | 4.00 | 1.50 | 1.50 |
| Stearyl alcohol | 12.00 | 16.00 | 18.00 |
| Cetyl alcohol | 6.00 | — | — |
| Cocoglycerides (mp 30-32° C., Novata AB) | — | 5.00 | 4.00 |
| PEG-20 glyceryl stearate | 5.00 | — | — |
| Ceteareth-30 | 3.00 | 3.00 | — |
| Ceteareth-12 | — | −50 | 3.00 |
| Perfume | 1.00 | 1.00 | 1.00 |
| Aluminum chlorohydrate | 20.00 | 20.00 | — |
| Al—Zr tetrachlorohydrex Gly* (USP) | — | — | 17.60 |
| Water of crystallization and glycine (if present) | — | — | 4.40 |
| Talc | 8.00 | 5.00 | 3.00 |
| Rumex acetosa leaf extract (0.5 wt % dry | 2.00 | 2.00 | 2.00 |
| extract in isopropyl myristate, | | | |
| extracted with isopropyl myristate | | | |
| L-Menthol | 0.20 | — | — |
| N-2,3-Trimethyl-2-isopropylbutamide | — | 0.20 | 0.20 |
| Cyclopentasiloxane | ad 100 | — | ad 100 |
| Dimethicone (5 cSt) | — | ad 100 | — |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A composition for personal hygiene or body care which is preferably a deodorant, antiperspirant, massage fluid, foot care agent, shaving auxiliary or oral care agent, comprising
0-7 wt % free water,
at least one cosmetic oil that is liquid under normal conditions,
an oily extract from the leaves of Rumex acetosa,
in addition to the abovementioned components, one or more cooling agents,
wherein all indications on quantities refer to the weight of the composition, without taking into account optionally present propellant.

2. The composition according to claim 1, wherein the extract is obtained by extracting leaves of Rumex acetosa with a cosmetic oil at a temperature of 5° C. to 30° C.

3. The composition according to claim 1, wherein the oily extract includes at least one oil that is liquid under normal conditions, and that is selected from the esters of linear or branched C₁-C₂₂ alkanols or C₁₄-C₂₂ alkenols or from the triesters of glycerin with linear or branched C₂-C₂₂ carboxylic acids, which may be saturated or unsaturated.

4. The composition according to claim 1, wherein the oily extract from the leaves of Rumex acetosa is included in a total amount of 0.0001-5 wt % relative to the total weight of dry extract in the composition, without taking into account optionally present propellant.

5. The composition according to claim 1, wherein the at least one cooling agent d) is selected from menthol, isopulegol, N-2,3-trimethyl-2-isopropylbutamide, menthyl lactate, menthylpyrrolidone carboxylic acid, menthyl methyl ether, menthoxypropane-1,2-diol, menthone glycerin acetal (9-methyl-6-(1-methylethyl)-1,4-dioxaspiro(4.5)decane-2-methanol), monomenthyl succinate, menthyl ethylamido oxalate (CAS-nr. 1122460-01-8), 2-hydroxy-methyl-3,5,5-trimethylcyclohexanol, 2-isopropyl-N,2,3-trimethylbutyramide (FEMA 3804), N-ethyl-p-menthane-3-carboxamide (FEMA 3455), in particular 1R,3R,4S—N-ethyl-p-menthane-3-carboxamide, ethyl 3-(p-menthane-3-carboxamido) acetate (FEMA 4309), (1R,2S,5R)-n-(4-methoxyphenyl)-p-menthane carboxamide (FEMA 4681), N-ethyl-2,2-diisopropylbutanamide (FEMA 4557), N-cyclopropyl-5-methyl-2-propane-2-yl-cyclohexane-1-carboxamide (FEMA 4693), N-(4-cyanomethylphenyl)-p-menthane carboxamide (FEMA 4496), N-(2-(pyridin-2-yl)ethyl)-3-p-menthane carboxamide (FEMA 4549), N-(2-hydroxyethyl)-2-isopropyl-2,3-dimethylbutanamide (FEMA 4602), N-(1,1-dimethyl-2-hydroxyethyl)-2,2-diethylbutanamide (FEMA 4603), (2S,5R)—N-[4-(2-amino-2-oxoethyl)phenyl]-p-menthane carboxamide (FEMA 4684), 2-[(2-p-menthoxy)-ethoxy]ethanol (FEMA 4718), (2,6-diethyl-5-isopropyl-2-methyltetrahydropyran (FEMA 4680), 3-(1-menthoxy)-2-methylpropane-1,2-diol (FEMA 3849), p-menthane-3,8-diol, in particular (+)-cis-p-menthane-3,8-diol und (−)-trans-p-menthane-3,8-diol as well as mixtures of (+)-cis-p-menthane-3,8-diol and (−)-trans-p-menthane-3,8-diol, in particular a mixture in the weight ratio 62:38 (FEMA 4053), (1R,3R,4S)-3-menthyl 3,6-dioxaheptanoate, (1R,2S,5R)-3-menthyl-methoxy acetate, (1R,2S,5R)-3-menthyl 3,6,9-trioxadecanoate, (1R,2S,5R)-3-menthyl 3,6,9-tri-oxadecanoate, (1R,2S,5R)-3-menthyl-(2-hydroxyethoxy) acetate, (1R,2S,5R)-menthyl 11-hydroxy-3,6,9-trioxaundecanoate, (−)-cubebol (FEMA 4497), N-(4-cyanomethylphenyl)-p-menthane carboxamide, N,N-dimethylmenthylsuccinamide(2-isopropyl-5-methylcyclohexyl 4-(dimethylamino)-4-oxobutanoate, FEMA 4230), 6-isopropyl-3,9-dimethyl-1,4-dioxaspiro[4.5]decane-2-one (FEMA 4285), and mixtures of such substances.

6. The composition according to claim 1, wherein the total amount of cooling agents d) is 0.01-5 wt %, referring to the weight of the composition without taking into account optionally present propellant.

7. The composition according to claim 1, wherein the weight ratio of cooling agent(s) to dry extract of the oily extracts from the leaves of Rumex acetosa is 0.01-200.

8. The composition according to claim 1, wherein the at least one cosmetic oil b) is comprised in a total amount of 1-95 wt %, referring to the weight of the composition without taking into account optionally present propellant.

9. The composition according to claim 1, further comprising at least one antiperspirant active substance selected from aluminum salts.

10. The composition according to claim 1, wherein the composition is packaged with a propellant and optionally additional active substances and/or auxiliaries in a spray can.

11. The composition according to claim 1, wherein the composition is a deodorant spray, comprising
a) 0-7 wt % of the free water,
b) in total 1-95 wt % of the at least one cosmetic oil that is liquid under normal conditions,
c) the oily extract from the leaves of Rumex acetosa in a total amount of 0.0001-5 wt % relative to the total weight of dry extract in the composition, and
d) the one or more cooling agents in a total amount of 0.01-5 wt %,
wherein the indications on quantities refer to the weight of the composition without taking into account optionally present propellant.

12. The composition according to claim 1, wherein the composition is an antiperspirant spray, comprising
a) 0-7 wt % of the free water,
b) in total 30-95 wt % of the at least one cosmetic oil that is liquid under normal conditions,
c) the oily extract from the leaves of Rumex acetosa in a total amount of 0.0001-5 wt % relative to the total weight of dry extract in the composition,
d) the one or more cooling agents in a total amount of 0.01-5 wt %, and
e) at least one antiperspirant active substance, selected from aluminum salts, in a total amount of 3-40 wt %, relative to the total weight of the active substance anhydrous (USP) in the total composition,
wherein the indications on quantities refer to the weight of the composition, without taking into account optionally present propellant.

13. The composition according to claim 1, wherein the composition is an antiperspirant wax stick, comprising:
a) 0-7 wt % of the free water,
b) in total 30-70 wt % of the at least one cosmetic oil that is liquid under normal conditions,
c) the oily extract from the leaves of Rumex acetosa in a total amount of 0.0001-5 wt % relative to the total weight of dry extract in the composition,
d) the one or more cooling agents in a total amount of 0.01-5 wt %,
e) 15-32 wt % of a fatty component that is solid under normal conditions of which more than 65 wt % relative to the total content of fatty components that are solid under normal conditions is C₁₆-C₃₀ fatty alcohols selected from the group consisting of stearyl alcohol, cetyl alcohol and mixtures thereof, and of which 0.1-3 wt %, relative to the whole stick, is one or more of arachidyl alcohol, behenyl alcohol, and at least one C₂₄-C₃₀ fatty alcohol,
f) 0.5-5 wt %, relative to the whole stick, of at least one fatty component with a melting point of 75-120° C. and 0.5-8 wt %, relative to the whole stick, of at least one fatty component with a melting point of 25-50° C.,
g) 0.5-30 wt % of at least one filler that is particularly preferably selected from the group consisting of talc, cellulose powder, starch and starch derivatives,
h) at least one antiperspirant active substance, selected from aluminum salts, in a total amount of 3-27 wt % relative to the total weight of the active substance anhydrous (USP) in the total composition,
wherein the indications on quantities refer to the weight of the composition.

14. The composition according to claim 1, wherein the composition is an antiperspirant wax stick, comprising:
a) 0-7 wt % of the free water,
b) in total 30-70 wt % of the at least one cosmetic oil that is liquid under normal conditions,
c) the oily extract from the leaves of Rumex acetosa in a total amount of 0.0001-5 wt % relative to the total weight of dry extract in the composition,
d) the one or more cooling agents in a total amount of 0.01-5 wt %,
e) 15-32 wt % of a fatty component that is solid under normal conditions of which more than 65 wt %, relative to the total content of fatty components that are solid under normal conditions, is C₁₆-C₃₀ fatty alcohols selected from the group consisting of stearyl alcohol, cetyl alcohol and mixtures thereof, and of which 0.1-3 wt %, relative to the whole stick, is one or more of arachidyl alcohol, behenyl alcohol, and at least one C₂₄-C₃₀ fatty alcohol,
f) 0.5-5 wt %, relative to the whole stick, of at least one fatty component with a melting point of 75-120° C., and 0.5-8 wt %, relative to the whole stick, of at least one fatty component with a melting point of 25-50° C.,
g) 0.5-30 wt % of at least one filler selected from the group consisting of talc, cellulose powder, starch and starch derivatives,
h) at least one antiperspirant active substance, selected from aluminum salts, in a total amount of 3-27 wt % relative to the total weight of the active substance anhydrous (USP) in the total composition and
i) 0.1-10 wt % relative to the whole stick, of at least one oil-in-water emulsifier,
wherein the indications on quantities refer to the weight of the composition.
